(19) United States
(12) Patent Application Publication
(10) Pub. No.: US 2024/0336613 A1
(43) Pub. Date: Oct. 10, 2024

(54) HETEROARYL COMPOUNDS FOR TREATING HUNTINGTON'S DISEASE

(71) Applicant: PTC THERAPEUTICS, INC., South Plainfield, NJ (US)

(72) Inventors: Suresh BABU, Pennington, NJ (US); Rauful ALAM, Middlesex, NJ (US); Anuradha BHATTACHARYYA, Edison, NJ (US); Guangming CHEN, Bridgewater, NJ (US); Matthew S. EASTWOOD, Somerset, NJ (US); Seyedmorteza HOSSEYNI, Highland Park, NJ (US); Yao JIANG, South Plainfield, NJ (US); Gary Mitchell KARP, Princeton Junction, NJ (US); Young-Choon MOON, Warren, NJ (US); Jana NARASIMHAN, Scotch Plains, NJ (US); Hongyu REN, Florence, NJ (US); Nadiya SYDORENKO, Princeton, NJ (US); Matthew G. WOLL, Dunellen, NJ (US); Nanjing ZHANG, Princeton, NJ (US)

(21) Appl. No.: 18/293,349

(22) PCT Filed: Jul. 29, 2022
(86) PCT No.: PCT/US2022/038870

§ 371 Date: Jan. 29, 2024

Related U.S. Application Data
(60) Provisional application No. 63/203,761, filed on Jul. 30, 2021.

(51) Int. Cl. C07D 487/04; A61K 31/5025

(52) U.S. Cl. CPC C07D 487/04; A61K 31/5025

(57) ABSTRACT

The present description relates to compounds, forms, and pharmaceutical compositions thereof and methods of using such compounds, forms, or compositions thereof for treating or ameliorating Huntington's disease. In particular, the present description relates to compounds of Formula (I), forms and pharmaceutical compositions thereof and methods of using such compounds, forms, or compositions thereof for treating or ameliorating Huntington's disease.

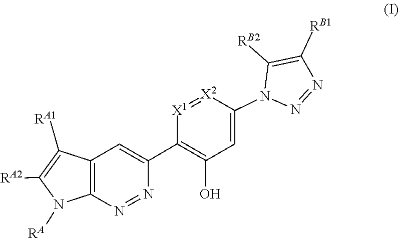

### CROSS-REFERENCE TO RELATED APPLICATIONS

This application is an International Application claiming benefit of U.S. Provisional Application No. 63/203,761 filed Jul. 30, 2021, the entirety of which is herein incorporated by reference.

### FIELD OF THE DISCLOSURE

An aspect of the present description relates to compounds, forms, and pharmaceutical compositions thereof and methods of using such compounds, forms, or compositions thereof useful for treating or ameliorating Huntington's disease. In particular, another aspect of the present description relates to substituted bicyclic heteroaryl compounds, forms and pharmaceutical compositions thereof and methods of using such compounds, forms, or compositions thereof for treating or ameliorating Huntington's disease.

### BACKGROUND

Huntington's disease (HD) is a progressive, autosomal dominant neurodegenerative disorder of the brain, having symptoms characterized by involuntary movements, cognitive impairment, and mental deterioration. Death, typically caused by pneumonia or coronary artery disease, usually occurs 13 to 15 years after the onset of symptoms. The prevalence of HD is between three and seven individuals per 100,000 in populations of western European descent. In North America, an estimated 30,000 people have HD, while an additional 200,000 people are at risk of inheriting the disease from an affected parent. The disease is caused by an expansion of uninterrupted trinucleotide CAG repeats in the “mutant” huntingtin (Htt) gene, leading to production of HTT (Htt protein) with an expanded poly-glutamine (polyQ) stretch, also known as a “CAG repeat” sequence. There are no current small molecule therapies targeting the underlying cause of the disease, leaving a high unmet need for medications that can be used for treating or ameliorating HD. Consequently, there remains a need to identify and provide small molecule compounds for treating or ameliorating HD.

All other documents referred to herein are incorporated by reference into the present application as though fully set forth herein.

### SUMMARY

An aspect of the present description relates to compounds of Formula (I):

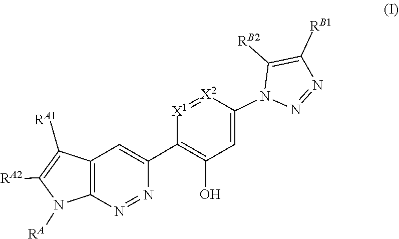

or a form thereof, wherein R^{A}, R^{A1}, R^{A2}, X¹, X², R^{B1}, and R^{B2} are as defined herein.

An aspect of the present description also relates to a method for use of a compound of Formula (I) or a form or composition thereof to treat or ameliorate HD in a subject in need thereof comprising, administering to the subject an effective amount of the compound or a form or composition thereof.

An aspect of the present description further relates to a use of a compound of Formula (I) or a form thereof to treat or ameliorate HD in a subject in need thereof comprising, administering to the subject an effective amount of the compound or a form thereof.

An aspect of the present description further relates to a use of a compound of Formula (I) or a form thereof for the preparation of a medicament useful to treat or ameliorate HD in a subject in need thereof comprising, administering to the subject an effective amount of the medicament.

An aspect of the present description further relates to a use of a compound of Formula (I) or a form thereof used in combination with other agents useful for treating or ameliorating HD in a subject in need thereof comprising, administering to the subject an effective amount of a combination product for treating or ameliorating HD.

### DETAILED DESCRIPTION

An aspect of the present description relates to compounds of Formula (I):

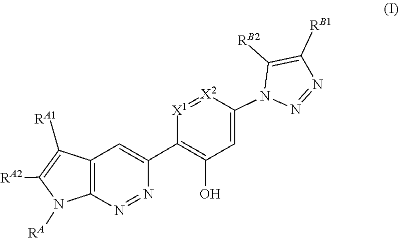

or a form thereof, wherein:

R^{A} is

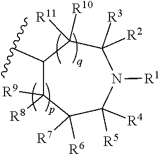

wherein, p and q are each independently 0 or 1;
X¹ is selected from the group consisting of CH, C-halogen, and N;
X² is selected from the group consisting of C-halogen and CF;
R¹ is selected from the group consisting of hydrogen, hydroxyl, and C₁₋₄alkyl;
R², R³, R⁴, R⁵, R⁶, R⁷, R⁸, R⁹, R¹⁰, and R¹¹ are each independently selected from selected from the group consisting of hydrogen, halogen, hydroxyl, cyano, C₁₋₄alkyl, deutero-C₁₋₄alkyl, halo-C₁₋₄alkyl, amino, C₁₋₄alkyl-amino, (C₁₋₄alkyl) 2-amino, C₁₋₄alkoxy, and halo-C₁₋₄alkoxy; or
R² and R³ together with the atom to which they are attached form a saturated 3-6 membered ring, incorporating 0 or 1 heteroatom ring members selected from N, O, and S; or
R² and R⁴ together with the atom to which they are attached form a saturated 5-10 membered ring system; or
R² and R⁷ together with the atom to which they are attached form a saturated 5-10 membered ring system; or
R⁴ and R⁵ together with the atom to which they are attached form a saturated 3-6 membered ring, incorporating 0 or 1 heteroatom ring members selected from N, O, and S;

R^{A1} and R^{A2} are each independently selected from the group consisting of hydrogen, deuterium, halogen, hydroxy, cyano, C₁₋₄alkyl, deutero-C₁₋₄alkyl, halo-C₁₋₄alkyl, C₁₋₄alkoxy, halo-C₁₋₄alkoxy, C₁₋₄alkoxy-C₁₋₄alkyl, amino, C₁₋₄alkyl-amino, (C₁₋₄alkyl)₂-amino, amino-C₁₋₄alkyl, and hydroxy-C₁₋₄alkyl; and
R^{B1} and R^{B2} are each independently selected from the group consisting of hydrogen, deuterium, halogen, hydroxyl, cyano, C₁₋₄alkyl, deutero-C₁₋₄alkyl, halo-C₁₋₄alkyl, C₁₋₄alkoxy, deutero-C₁₄alkoxy, and halo-C₁₋₄alkoxy;
wherein the form of the compound is selected from the group consisting of salt, racemate, enantiomer, diastereomer, stereoisomer, and tautomer form thereof.

### Aspects of the Description

One aspect of the present description relates to compounds of Formula (I):

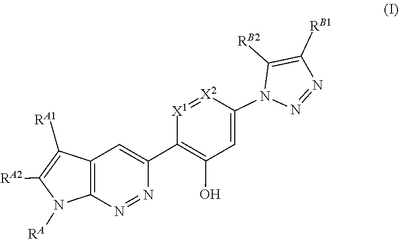

or a form thereof, wherein:

R^{A} is

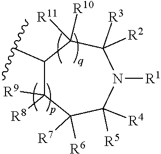

wherein, p and q are each independently 0 or 1;
X¹ is selected from the group consisting of CH, C-halogen, and N;
X² is selected from the group consisting of CH and C-halogen;
R¹ is selected from the group consisting of hydrogen, hydroxyl, and C₁₋₄alkyl;
R², R³, R⁴, R⁵, R⁶, R⁷, R⁸, R⁹, R¹⁰, and R¹¹ are each independently selected from selected from the group consisting of hydrogen, halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy; or
R² and R³ together with the atom to which they are attached form a saturated 3-6 membered ring, incorporating 0 or 1 heteroatom ring members selected from N, O, and S; or
R² and R⁴ together with the atom to which they are attached form a saturated 5-10 membered ring system; or
R² and R⁷ together with the atom to which they are attached form a saturated 5-10 membered ring system; or
R⁴ and R⁵ together with the atom to which they are attached form a saturated 3-6 membered ring, incorporating 0 or 1 heteroatom ring members selected from N, O, and S;
R^{A1} and R^{A2} are each independently selected from the group consisting of hydrogen, deuterium, halogen, hydroxy, cyano, C₁₋₄alkyl, deutero-C₁₄alkyl, halo-C₁₋₄alkyl, C₁₋₄alkoxy, halo-C₁₋₄alkoxy, C₁₋₄alkoxy-C₁₋₄alkyl, amino, C₁₋₄alkylamino, (C₁₋₄alkyl)₂amino, amino-C₁₋₄alkyl, and hydroxy-C₁₋₄alkyl; and
R^{B1} and R^{B2} are each independently selected from the group consisting of hydrogen, deuterium, halogen, hydroxyl, cyano, C₁₋₄alkyl, deutero-C₁₋₄alkyl, halo-C₁₋₄alkyl, C₁₋₄alkoxy, deutero-C₁₋₄alkoxy, and halo-C₁₋₄alkoxy;
wherein the form of the compound is selected from the group consisting of a salt, racemate, enantiomer, diastereomer, stereoisomer, and tautomer form thereof.

One aspect includes a compound of Formula (I), wherein R^{A} is:

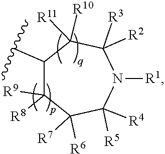

wherein p and q are each independently 0 or 1.

One aspect includes a compound of Formula (I), wherein R^{A} is:

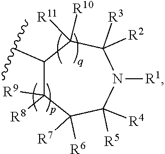

wherein p and q are 0.

One aspect includes a compound of Formula (I), wherein R^{A} is:

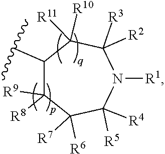

wherein p is 0 and q is 1

One aspect includes a compound of Formula (I), wherein R^{A} is:

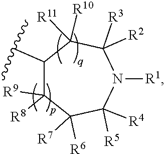

wherein p is 1 and q is 0.

One aspect includes a compound of Formula (I), wherein R^{A} is:

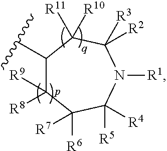

wherein p and q are 1.

Another aspect includes a compound of Formula (I), wherein R^{A} is selected from the group consisting of:

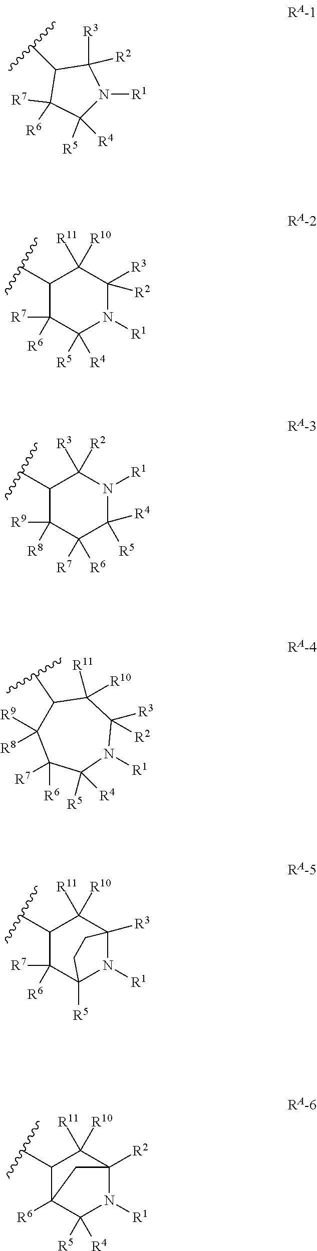

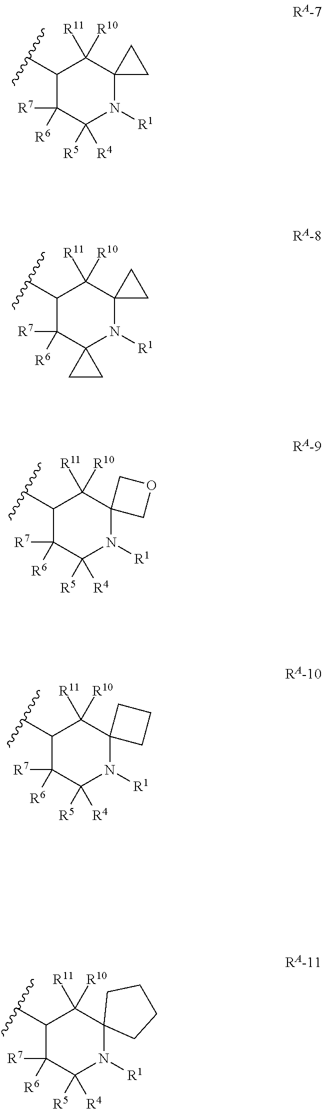

and any stereoisomer thereof.

Another aspect includes a compound of Formula (I), wherein R^{A} is:

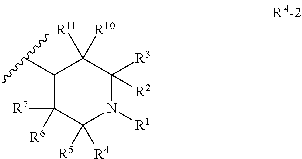

and any stereoisomer thereof.

Another aspect includes a compound of Formula (I), wherein R^{A} is:

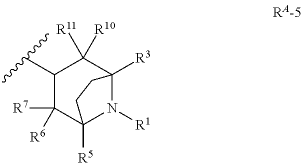

and any stereoisomer thereof.

Another aspect includes a compound of Formula (I), wherein R^{A} is:

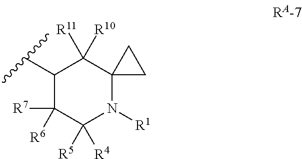

and any stereoisomer thereof.

Another aspect includes a compound of Formula (I), wherein R^{A} is:

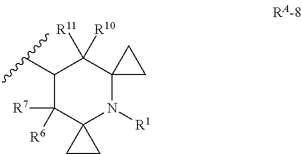

and any stereoisomer thereof.

Another aspect includes a compound of Formula (I), wherein R^{A} is:

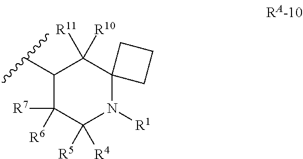

and any stereoisomer thereof.

Another aspect includes a compound of Formula (I), wherein R^{A} is:

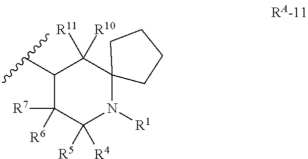

and any stereoisomer thereof.

Another aspect includes a compound of Formula (I), wherein R^{A}-2 comprises:

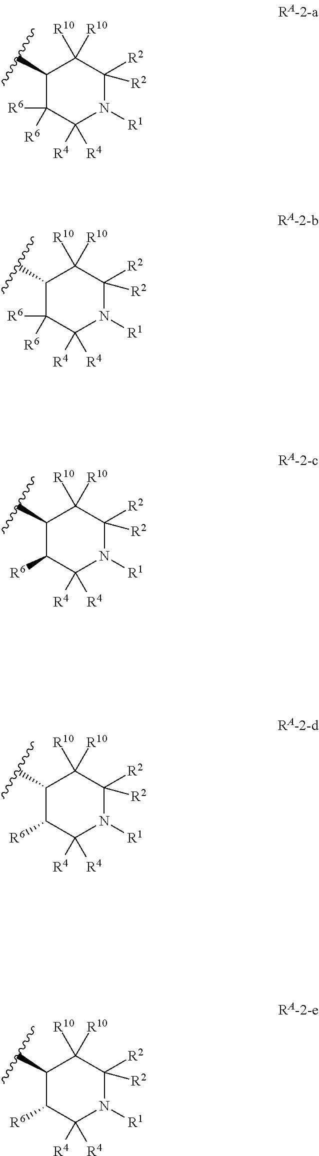

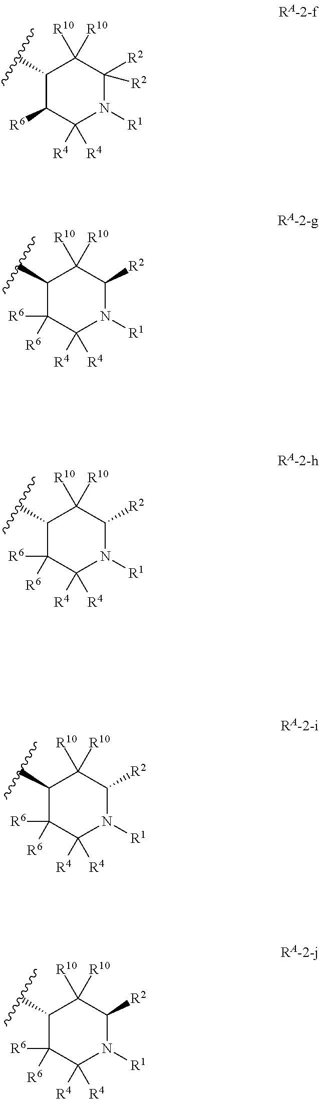

or any additional stereoisomers thereof.

Another aspect includes a compound of Formula (I), wherein R^{A} is:

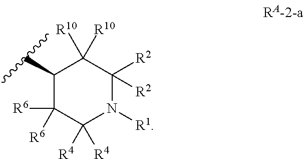

Another aspect includes a compound of Formula (I), wherein R^{A} is:

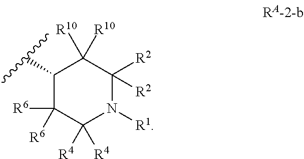

Another aspect includes a compound of Formula (I), wherein R^{A} is:

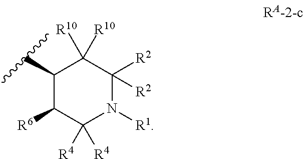

Another aspect includes a compound of Formula (I), wherein R^{A} is:

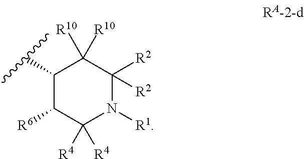

Another aspect includes a compound of Formula (I), wherein R^{A} is:

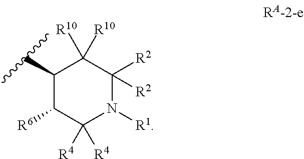

Another aspect includes a compound of Formula (I), wherein R^{A} is:

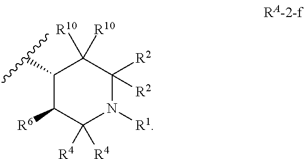

Another aspect includes a compound of Formula (I), wherein R^{A}-2-c is:

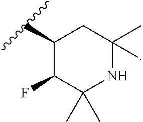

Another aspect includes a compound of Formula (I), wherein R^{A}-2-d is:

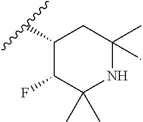

Another aspect includes a compound of Formula (I), wherein R^{A}-5 comprises:

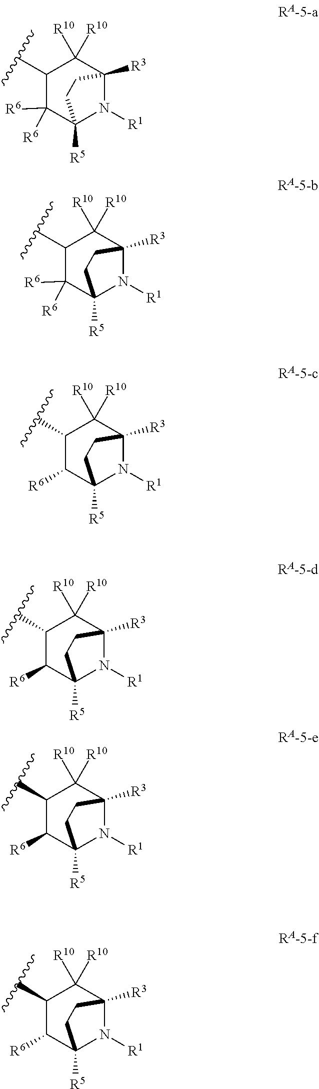

or any additional stereoisomers thereof.

Another aspect includes a compound of Formula (I), wherein R^{A} is:

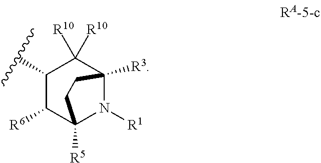

Another aspect includes a compound of Formula (I), wherein R^{A} is:

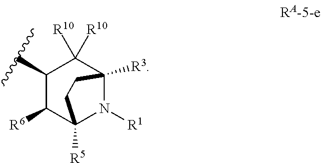

Another aspect includes a compound of Formula (I), wherein R^{A}-7 comprises:

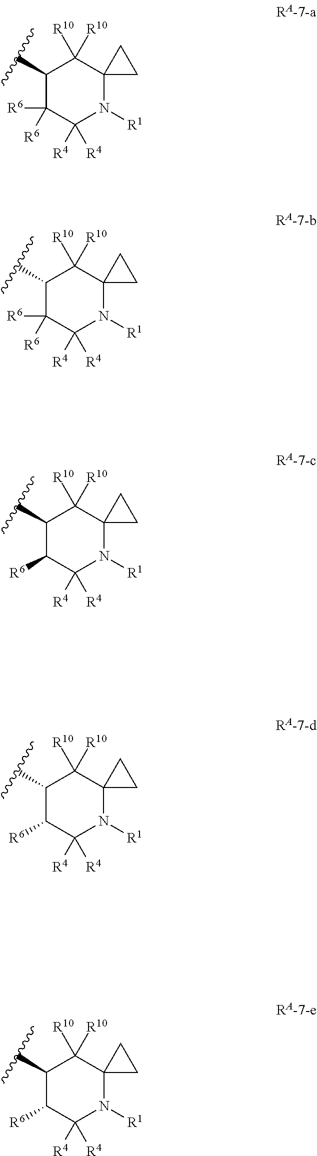

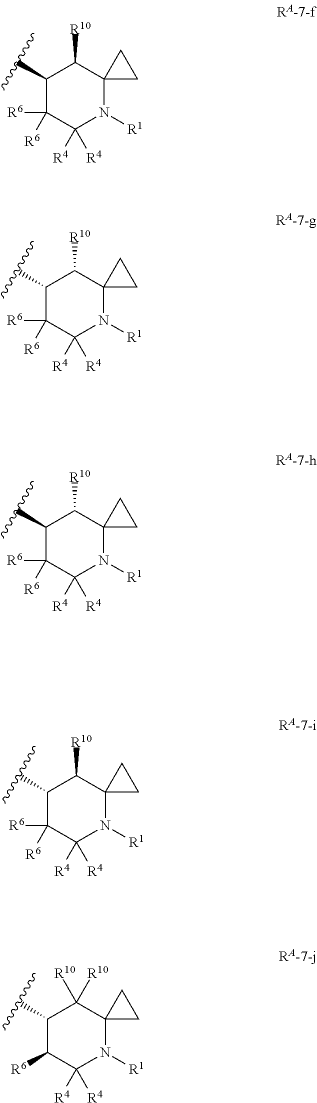

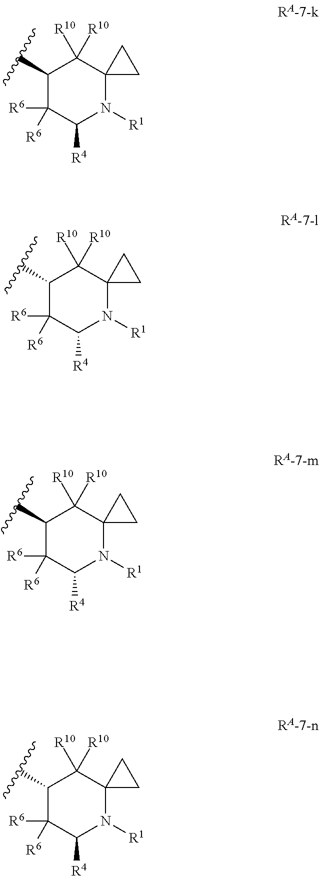

or any additional stereoisomers thereof.

Another aspect includes a compound of Formula (I), wherein R^{A} is:

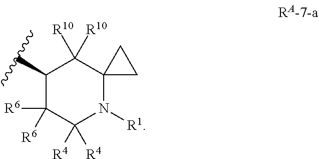

Another aspect includes a compound of Formula (I), wherein R^{A} is:

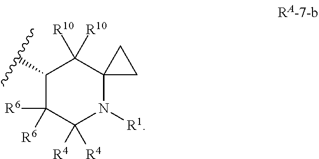

Another aspect includes a compound of Formula (I), wherein R^{A}-7-a is:

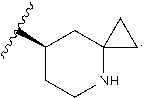 [SMILES: *[C@@H]1CCNC2(CC2)C1]

Another aspect includes a compound of Formula (I), wherein R^{A}-7-b is:

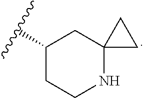 [SMILES: *[C@H]1CCNC2(CC2)C1]

Another aspect includes a compound of Formula (I), wherein R^{A}-8 comprises:

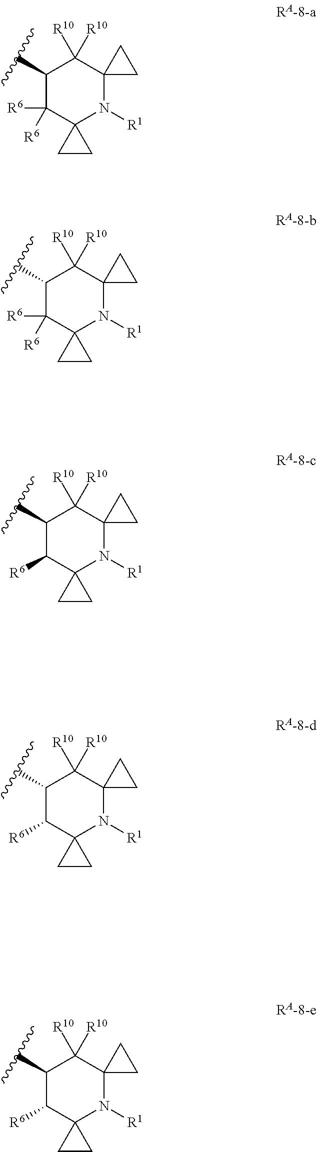

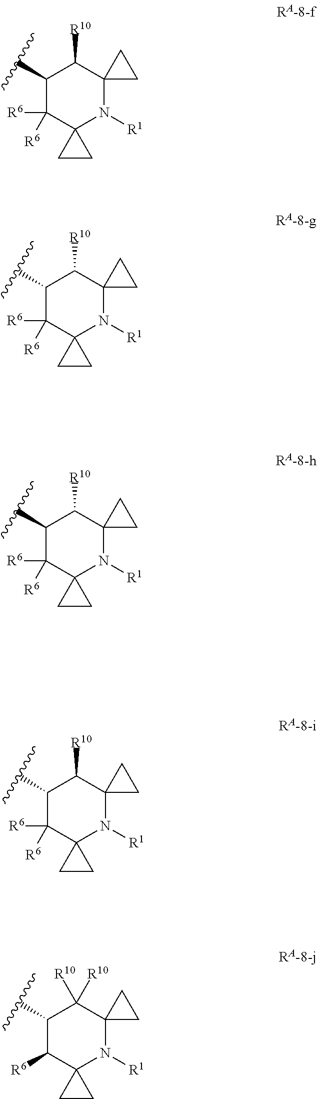

or any additional stereoisomers thereof.

Another aspect includes a compound of Formula (I), wherein R^{A}-10 comprises:

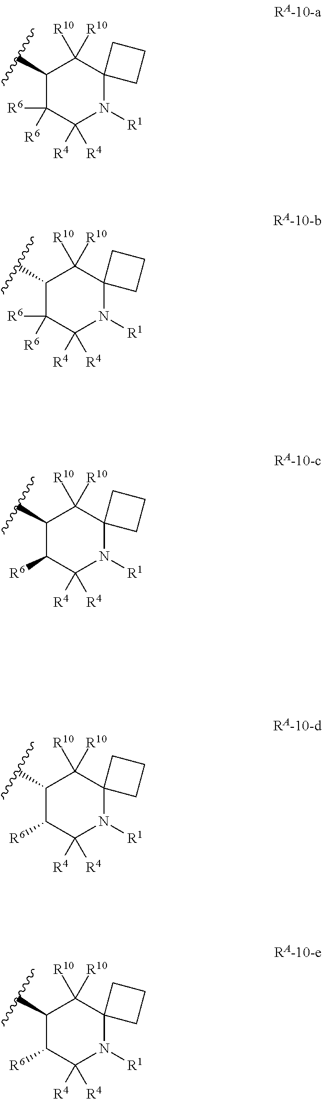

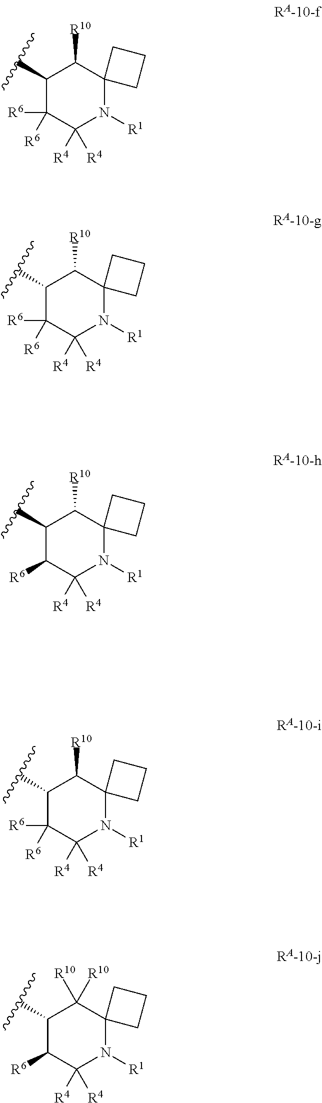

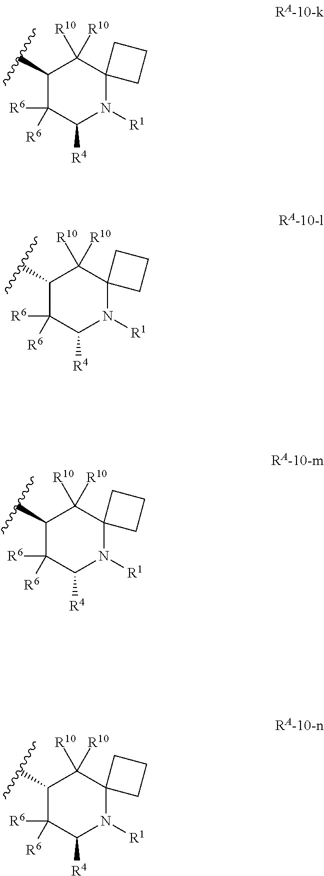

or any additional stereoisomers thereof.

Another aspect includes a compound of Formula (I), wherein R^{A} is:

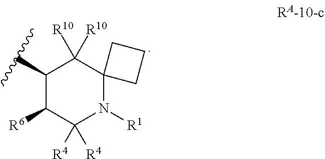

Another aspect includes a compound of Formula (I), wherein R^{A} is:

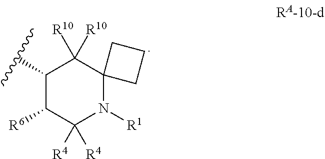

Another aspect includes a compound of Formula (I), wherein R^{A}-11 comprises:

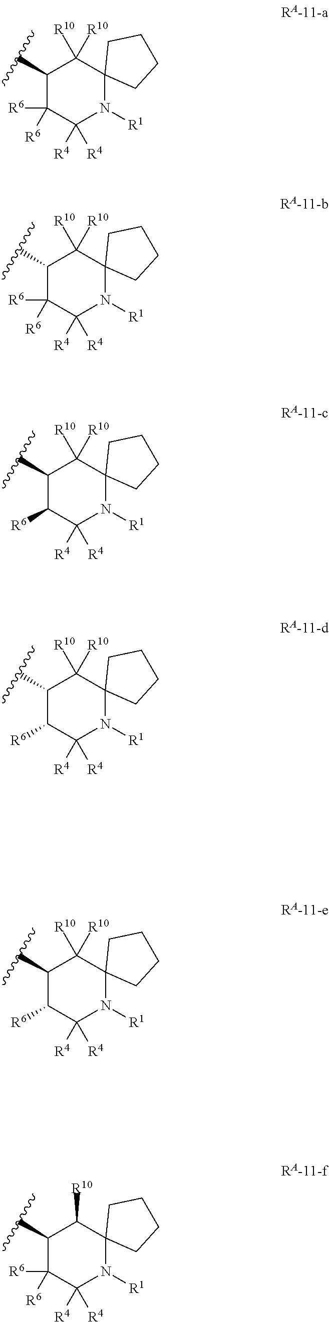

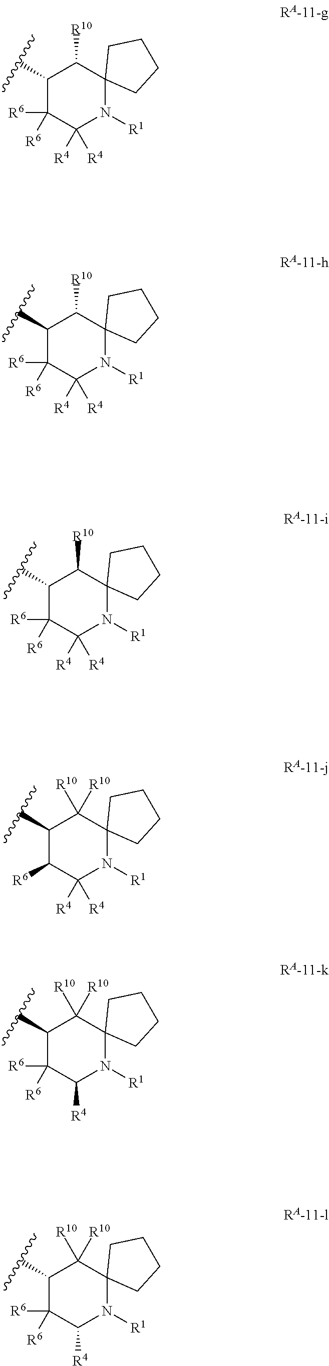

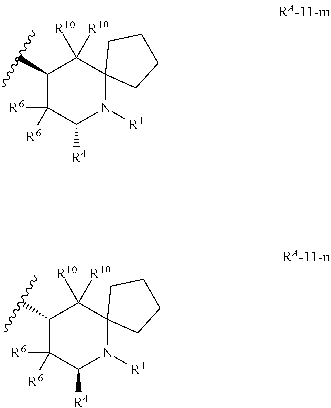

or any additional stereoisomers thereof.

Another aspect includes a compound of Formula (I), wherein R^{A} is:

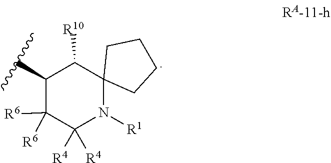

Another aspect includes a compound of Formula (I), wherein R^{A} is:

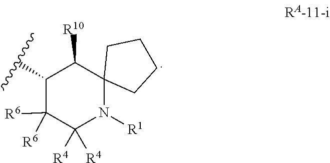

One aspect includes a compound of Formula (I), wherein X¹ is selected from the group consisting of CH, C-halogen, and N.

Another aspect includes a compound of Formula (I), wherein X¹ is CH.

Another aspect includes a compound of Formula (I), wherein X¹ is C-halogen, wherein halogen is selected from the group consisting of bromo, chloro, fluoro, and iodo.

Another aspect includes a compound of Formula (I), wherein X¹ is C-F.

Another aspect includes a compound of Formula (I), wherein X¹ is N.

One aspect includes a compound of Formula (I), wherein X² is selected from the group consisting of CH and C-halogen.

Another aspect includes a compound of Formula (I), wherein X² is CH.

Another aspect includes a compound of Formula (I), wherein X² is C-halogen, wherein halogen is selected from the group consisting of bromo, chloro, fluoro, and iodo.

Another aspect includes a compound of Formula (I), wherein X² is CF.

One aspect includes a compound of Formula (I), wherein R¹ is selected from the group consisting of hydrogen, hydroxyl, and C₁₋₄alkyl.

Another aspect includes a compound of Formula (I), wherein R¹ is selected from the group consisting of hydrogen and C₁₋₄alkyl.

Another aspect includes a compound of Formula (I), wherein R¹ is hydrogen.

Another aspect includes a compound of Formula (I), wherein R¹ is hydroxyl.

Another aspect includes a compound of Formula (I), wherein R¹ is C₁₋₄alkyl.

Another aspect includes a compound of Formula (I), wherein R¹ is C₁₋₄alkyl selected from the group consisting of methyl, ethyl, propyl, isopropyl, butyl, and tert-butyl.

Another aspect includes a compound of Formula (I), wherein R¹ is methyl.

One aspect includes a compound of Formula (I), wherein R², R³, R⁴, R⁵, R⁶, R⁷, R⁸, R⁹, R¹⁰, and R¹¹ are each independently selected from the group consisting of hydrogen, halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy.

Another aspect includes a compound of Formula (I), wherein R², R³, R⁴, R⁵, R⁶, R⁷, R⁸, R⁹, R¹⁰, and R¹¹ are each independently selected from the group consisting of hydrogen, halogen, and C₁₋₄alkyl.

Another aspect includes a compound of Formula (I), wherein R², R³, R⁴, R⁵, R⁶, R⁷, R⁸, R⁹, R¹⁰, and R¹¹ are each independently hydrogen.

Another aspect includes a compound of Formula (I), wherein R², R³, R⁴, R⁵, R⁶, R⁷, R⁸, R⁹, R¹⁰, and R¹¹ are each independently C₁₋₄alkyl selected from the group consisting of methyl, ethyl, propyl, isopropyl, butyl, and tert-butyl.

Another aspect includes a compound of Formula (I), wherein R², R³, R⁴, R⁵, R⁶, R⁷, R⁸, R⁹, R¹⁰, and R¹¹ are each independently methyl.

Another aspect includes a compound of Formula (I), wherein R² and R³ are each independently C₁₋₄alkyl selected from the group consisting of methyl, ethyl, propyl, isopropyl, butyl, and tert-butyl.

Another aspect includes a compound of Formula (I), wherein R² is methyl.

Another aspect includes a compound of Formula (I), wherein R³ is methyl.

Another aspect includes a compound of Formula (I), wherein R² and R³ are each methyl.

Another aspect includes a compound of Formula (I), wherein R⁴ and R⁵ are each independently C₁₋₄alkyl selected from the group consisting of methyl, ethyl, propyl, isopropyl, butyl, and tert-butyl.

Another aspect includes a compound of Formula (I), wherein R⁴ is methyl.

Another aspect includes a compound of Formula (I), wherein R⁵ is methyl.

Another aspect includes a compound of Formula (I), wherein R⁴ and R⁵ are each methyl.

Another aspect includes a compound of Formula (I), wherein R², R³, R⁴, R⁵, R⁶, R⁷, R⁸, R⁹, R¹⁰, and R¹¹ are each independently halogen selected from the group consisting of bromo, chloro, fluoro, and iodo.

Another aspect includes a compound of Formula (I), wherein R², R³, R⁴, R⁵, R⁶, R⁷, R⁸, R⁹, R¹⁰, and R¹¹ are each independently fluoro.

Another aspect includes a compound of Formula (I), wherein R⁷, R⁸, R¹⁰, and R¹¹ are each independently halogen selected from the group consisting of bromo, chloro, fluoro, and iodo.

Another aspect includes a compound of Formula (I), wherein R⁷, R⁸, R¹⁰, and R¹¹ are each independently fluoro.

Another aspect includes a compound of Formula (I), wherein R⁷ is fluoro.

Another aspect includes a compound of Formula (I), wherein R⁸ is fluoro.

Another aspect includes a compound of Formula (I), wherein R¹º is fluoro.

Another aspect includes a compound of Formula (I), wherein R¹¹ is fluoro.

One aspect includes a compound of Formula (I), wherein R² and R³ together with the atom to which they are attached form a saturated 3-6 membered ring, incorporating 0 or 1 heteroatom ring members selected from N, O, and S.

Another aspect includes a compound of Formula (I), wherein R² and R³ form a cyclopropane ring.

Another aspect includes a compound of Formula (I), wherein R² and R³ form a cycobutane ring.

Another aspect includes a compound of Formula (I), wherein R² and R³ form a cyclopentane ring.

One aspect includes a compound of Formula (I), wherein R² and R⁴ together with the atom to which they are attached form a saturated 5-10 membered ring system.

One aspect includes a compound of Formula (I), wherein R² and R⁷ together with the atom to which they are attached form a saturated 5-10 membered ring system.

One aspect includes a compound of Formula (I), wherein R⁴ and R⁵ together with the atom to which they are attached form a saturated 3-6 membered ring, incorporating 0 or 1 heteroatom ring members selected from N, O, and S.

Another aspect includes a compound of Formula (I), wherein R⁴ and R⁵ form a cyclopropane ring.

Another aspect includes a compound of Formula (I), wherein R⁴ and R⁵ form a cycobutane ring.

Another aspect includes a compound of Formula (I), wherein R⁴ and R⁵ form a cyclopentane ring.

One aspect includes a compound of Formula (I), wherein R^{A1} and R^{A2} are each independently selected from the group consisting of hydrogen, deuterium, halogen, hydroxy, cyano, C₁₋₄alkyl, deutero-C₁₋₄alkyl, halo-C₁₋₄alkyl, C₁₋₄alkoxy, halo-C₁₋₄alkoxy, C₁₋₄alkoxy-C₁₋₄alkyl, amino, C₁₋₄alkyl-amino, (C₁₋₄alkyl)₂-amino, amino-C₁₋₄alkyl, and hydroxy-C₁₋₄alkyl.

Another aspect includes a compound of Formula (I), wherein R^{A1} and R^{A2} are each independently selected from the group consisting of hydrogen and C₁₋₄alkyl.

Another aspect includes a compound of Formula (I), wherein R^{A1} and R^{A2} are each independently hydrogen.

Another aspect includes a compound of Formula (I), wherein R^{A1} and R^{A2} are each independently C₁₋₄alkyl selected from the group consisting of methyl, ethyl, propyl, isopropyl, butyl, and tert-butyl.

Another aspect includes a compound of Formula (I), wherein R^{A1} and R^{A2} are each independently methyl.

Another aspect includes a compound of Formula (I), wherein R^{A1} is selected from the group consisting of hydrogen, deuterium, halogen, hydroxy, cyano, C₁₋₄alkyl, deutero-C₁₋₄alkyl, halo-C₁₋₄alkyl, C₁₋₄alkoxy, halo-C₁₋₄alkoxy, C₁₋₄alkoxy-C₁₋₄alkyl, amino, C₁₋₄alkyl-amino, (C₁₋₄alkyl)₂-amino, amino-C₁₋₄alkyl, and hydroxy-C₁₋₄alkyl.

Another aspect includes a compound of Formula (I), wherein R^{A1} is selected from the group consisting of hydrogen, deuterium, halogen, cyano, C₁₋₄alkyl, deutero-C₁₋₄alkyl, and halo-C₁₋₄alkyl.

Another aspect includes a compound of Formula (I), wherein R^{A1} is hydrogen.

Another aspect includes a compound of Formula (I), wherein R^{A2} is selected from the group consisting of hydrogen, deuterium, halogen, hydroxy, cyano, C₁₋₄alkyl, deutero-C₁₋₄alkyl, halo-C₁₋₄alkyl, C₁₋₄alkoxy, halo-C₁₋₄alkoxy, C₁₋₄alkoxy-C₁₋₄alkyl, amino, C₁₋₄alkyl-amino, (C₁₋₄alkyl)₂-amino, amino-C₁₋₄alkyl, and hydroxy-C₁₋₄alkyl.

Another aspect includes a compound of Formula (I), wherein R^{A2} is selected from the group consisting of hydrogen and C₁₋₄alkyl.

Another aspect includes a compound of Formula (I), wherein R^{A2} is hydrogen.

Another aspect includes a compound of Formula (I), wherein R^{A2} is C₁₋₄alkyl selected from the group consisting of methyl, ethyl, propyl, isopropyl, butyl, and tert-butyl.

Another aspect includes a compound of Formula (I), wherein R^{A2} is methyl.

One aspect includes a compound of Formula (I), wherein RBI and R^{B2} are each independently selected from the group consisting of hydrogen, deuterium, halogen, hydroxyl, cyano, C₁₋₄alkyl, deutero-C₁₋₄alkyl, halo-C₁₋₄alkyl, C₁₋₄alkoxy, deutero-C₁₋₄alkoxy, and halo-C₁₋₄alkoxy.

Another aspect includes a compound of Formula (I), wherein R^{B1} and R^{B2} are each independently selected from the group consisting of hydrogen, halogen, and C₁₋₄alkyl.

Another aspect includes a compound of Formula (I), wherein R^{B1} and R^{B2} are each independently hydrogen.

Another aspect includes a compound of Formula (I), wherein R^{B1} is hydrogen.

Another aspect includes a compound of Formula (I), wherein R^{B2} is hydrogen.

Another aspect includes a compound of Formula (I), wherein R^{B1} and R^{B2} are each independently C₁₋₄alkyl, wherein C₁₋₄alkyl is selected from the group consisting of methyl, ethyl, propyl, isopropyl, butyl, and tert-butyl.

Another aspect includes a compound of Formula (I), wherein R^{B1} and R^{B2} are each independently methyl.

Another aspect includes a compound of Formula (I), wherein R^{B1} is methyl.

Another aspect includes a compound of Formula (I), wherein R^{B2} is methyl.

Another aspect includes a compound of Formula (I), wherein R^{B1} and R^{B2} are each independently halogen, wherein halogen is selected from the group consisting of bromo, chloro, fluoro, and iodo.

Another aspect includes a compound of Formula (I), wherein R^{B1} and R^{B2} are each independently chloro.

Another aspect includes a compound of Formula (I), wherein R^{B1} is chloro.

Another aspect of the compound of Formula (I) is a compound of Formula (Ia):

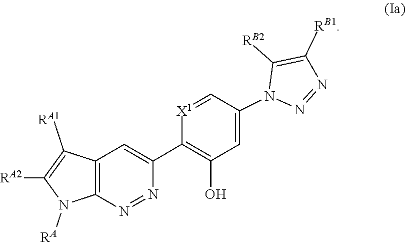

Another aspect of the compound of Formula (I) is a compound of Formula (Ib):

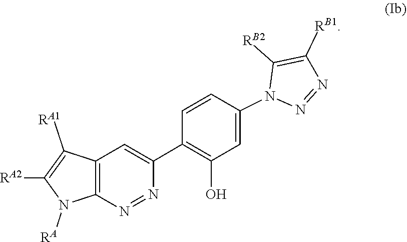

An aspect of the compound of Formula (I) or a form thereof includes a compound selected from the group consisting of the following, wherein “^{#}” indicates that the compound is a racemic mixture of enantiomers:

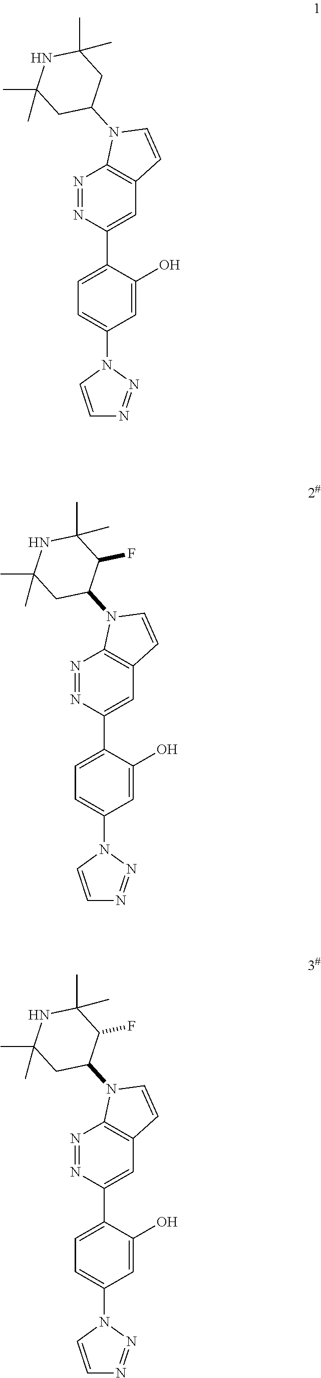

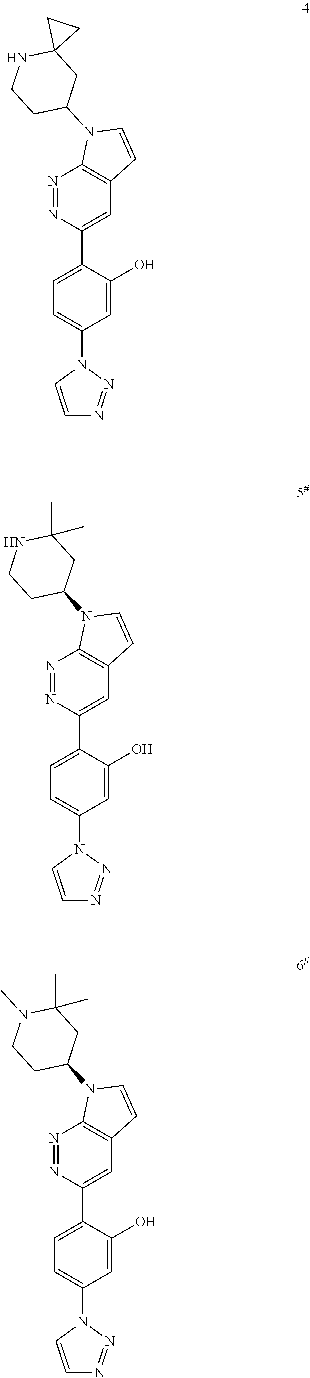 [SMILES: Oc1cc(-n2ccnn2)ccc1-c1cc2ccn(C3CCNC4(CC4)C3)c2nn1 ; CC1(C)C[C@@H](n2ccc3cc(-c4ccc(-n5ccnn5)cc4O)nnc32)CCN1 ; CN1CC[C@H](n2ccc3cc(-c4ccc(-n5ccnn5)cc4O)nnc32)CC1(C)C]

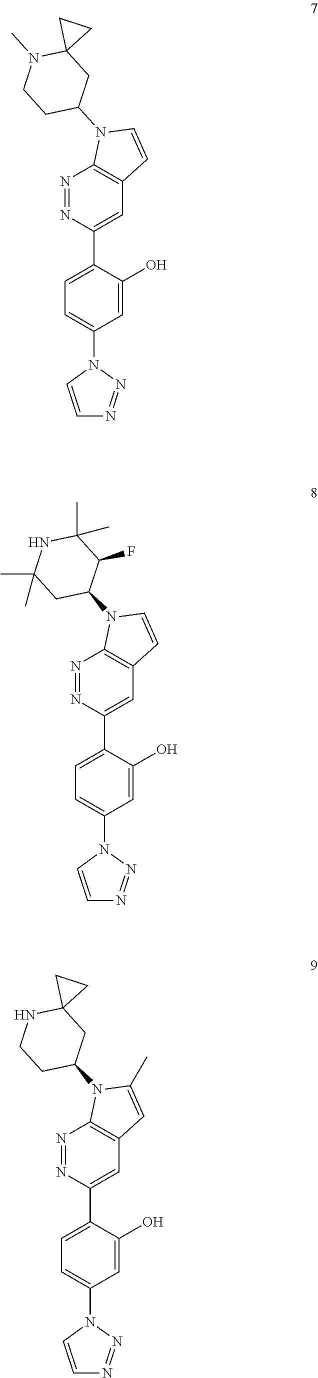 [SMILES: CN1CCC(n2ccc3cc(-c4ccc(-n5ccnn5)cc4O)nnc32)CC12CC2 ; CC1(C)C[C@H](n2ccc3cc(-c4ccc(-n5ccnn5)cc4O)nnc32)[C@H](F)C(C)(C)N1 ; Cc1cc2cc(-c3ccc(-n4ccnn4)cc3O)nnc2n1[C@H]1CCNC2(CC2)C1]

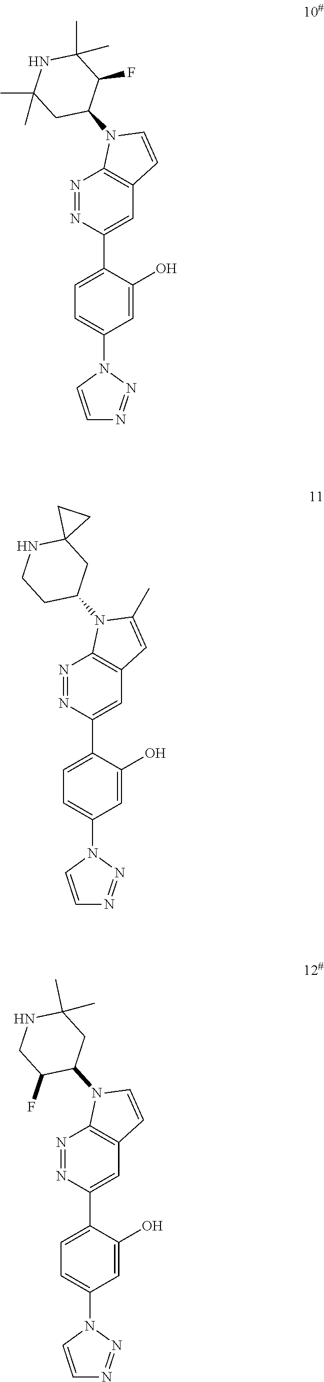

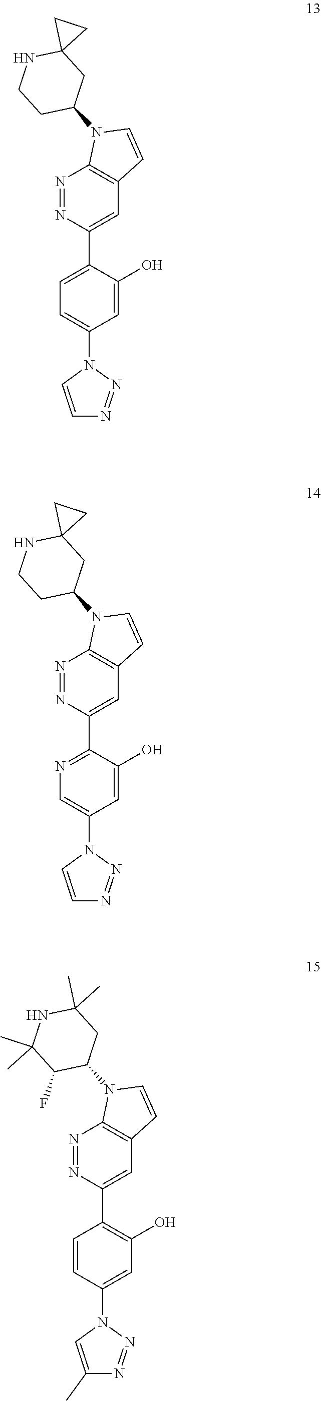 [SMILES: Oc1cc(-n2ccnn2)ccc1-c1cc2ccn([C@H]3CCNC4(CC4)C3)c2nn1 ; Oc1cc(-n2ccnn2)cnc1-c1cc2ccn([C@H]3CCNC4(CC4)C3)c2nn1 ; Cc1cn(-c2ccc(-c3cc4ccn([C@H]5CC(C)(C)NC(C)(C)[C@H]5F)c4nn3)c(O)c2)nn1]

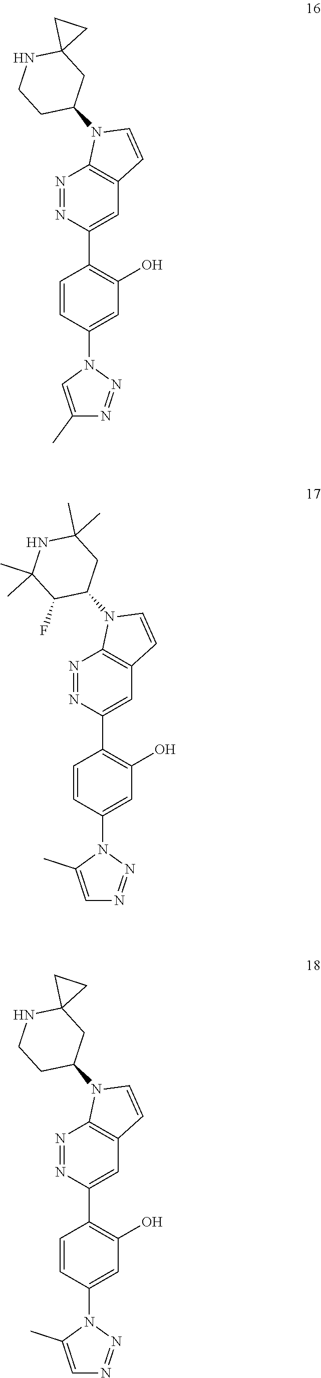 [SMILES: Cc1cn(-c2ccc(-c3cc4ccn([C@H]5CCNC6(CC6)C5)c4nn3)c(O)c2)nn1 ; Cc1cnnn1-c1ccc(-c2cc3ccn([C@H]4CC(C)(C)NC(C)(C)[C@H]4F)c3nn2)c(O)c1 ; Cc1cnnn1-c1ccc(-c2cc3ccn([C@H]4CCNC5(CC5)C4)c3nn2)c(O)c1]

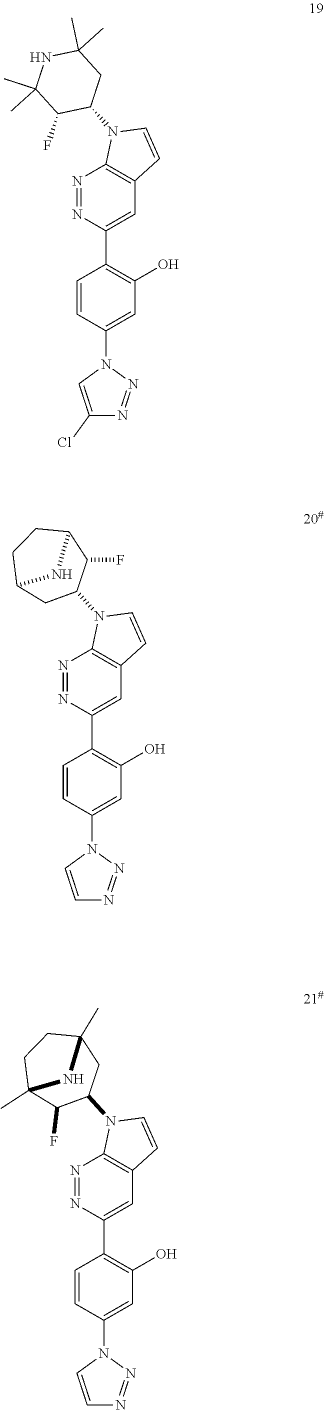

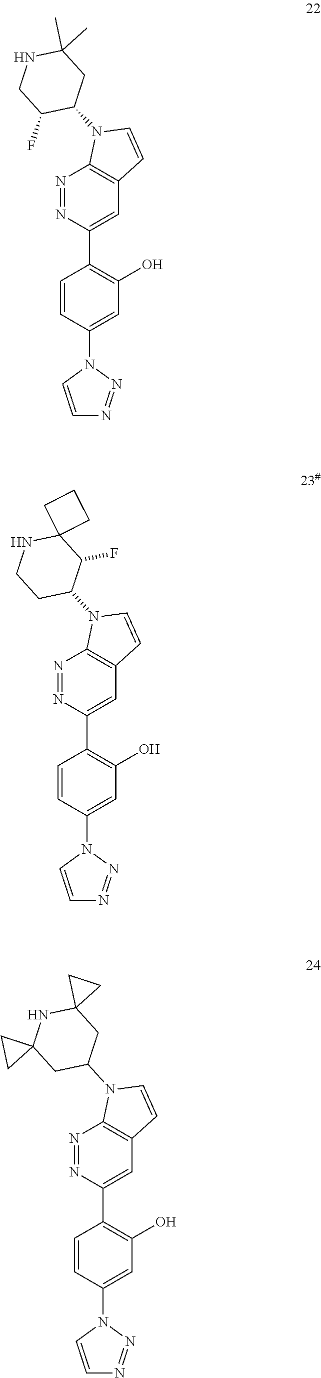

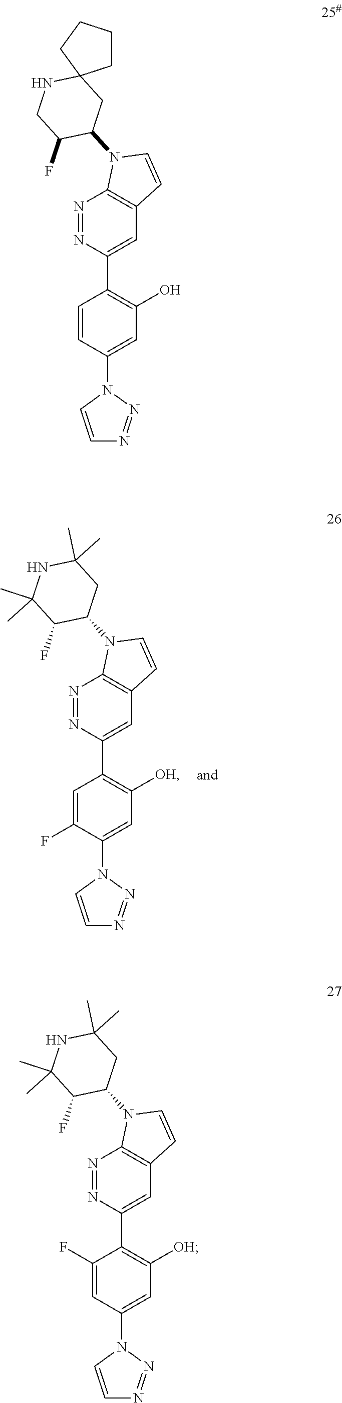

wherein the form of the compound is selected from the group consisting of a salt, hydrate, enantiomer, diastereomer, stereoisomer, and tautomer form thereof.

An aspect the compound of Formula (I) or a form thereof (wherein compound number (#\hat{ }) indicates that the salt form was isolated) includes a compound selected from the group consisting of:

| Cpd | Name |
|---|---|
| 1\hat{ } | 2-[7-(2,2,6,6-tetramethylpiperidin-4-yl)-7H-pyrrolo[2,3-c]pyridazin-3-yl]-5-(1H-1,2,3- |
| | triazol-1-yl)phenol |
| 2\hat{ } | 2-{7-[(3S,4S)-3-fluoro-2,2,6,6-tetramethylpiperidin-4-yl]-7H-pyrrolo[2,3-c]pyridazin-3- |
| | yl}-5-(1H-1,2,3-triazol-1-yl)phenol and |
| | 2-{7-[(3R,4R)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol |
| 3\hat{ } | 2-{7-[(3R,4S)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol and |
| | 2-{7-[(3S,4R)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol |
| 4\hat{ } | 2-[7-(4-azaspiro[2.5]octan-7-yl)-7H-pyrrolo[2,3-c]pyridazin-3-yl]-5-(1H-1,2,3-triazol- |
| | 1-yl)phenol |
| 5\hat{ } | 2-{7-[(4RS)-2,2-dimethylpiperidin-4-yl]-7H-pyrrolo[2,3-c]pyridazin-3-yl}-5-(1H-1,2,3- |
| | triazol-1-yl)phenol |
| 6\hat{ } | 5-(1H-1,2,3-triazol-1-yl)-2-{7-[(4RS)-1,2,2-trimethylpiperidin-4-yl]-7H-pyrrolo[2,3- |
| | c]pyridazin-3-yl}phenol |
| 7\hat{ } | 2-[7-(4-methyl-4-azaspiro[2.5]octan-7-yl)-7H-pyrrolo[2,3-c]pyridazin-3-yl]-5-(1H- |
| | 1,2,3-triazol-1-yl)phenol |
| 8\hat{ } | 2-{7-[(3S,4S)-3-fluoro-2,2,6,6-tetramethylpiperidin-4-yl]-7H-pyrrolo[2,3-c]pyridazin-3- |
| | yl}-5-(1H-1,2,3-triazol-1-yl)phenol) |
| 9\hat{ } | 2-[7-(4-azaspiro[2.5]octan-7-yl)-6-methyl-7H-pyrrolo[2,3-c]pyridazin-3-yl]-5-(1H- |
| | 1,2,3-triazol-1-yl)phenol |
| 10\hat{ } | 2-{7-[(3S,4S)-3-fluoro-2,2,6,6-tetramethylpiperidin-4-yl]-6-methyl-7H-pyrrolo[2,3- |
| | c]pyridazin-3-yl}-5-(1H-1,2,3-triazol-1-yl)phenol |
| 11 | 2-{7-[(7R)-4-azaspiro[2.5]octan-7-yl]-7H-pyrrolo[2,3-c]pyridazin-3-yl}-5-(1H-1,2,3- |
| | triazol-1-yl)phenol |
| 12\hat{ } | 2-{7-[(4S,5R)-5-fluoro-2,2-dimethylpiperidin-4-yl]-7H-pyrrolo[2,3-c]pyridazin-3-yl}-5- |
| | (1H-1,2,3-triazol-1-yl)phenol and |
| | 2-{7-[(4R,5S)-5-fluoro-2,2-dimethylpiperidin-4-yl]-7H-pyrrolo[2,3-c]pyridazin-3-yl}-5- |
| | (1H-1,2,3-triazol-1-yl)phenol |
| 13\hat{ } | 2-{7-[(7S)-4-azaspiro[2.5]octan-7-yl]-7H-pyrrolo[2,3-c]pyridazin-3-yl}-5-(1H-1,2,3- |
| | triazol-1-yl)phenol |
| 14\hat{ } | 2-{7-[(7S)-4-azaspiro[2.5]octan-7-yl]-7H-pyrrolo[2,3-c]pyridazin-3-yl}-5-(1H-1,2,3- |
| | triazol-1-yl)pyridine-3-ol |
| 15\hat{ } | 2-{7-[(3S,4S)-3-fluoro-2,2,6,6-tetramethylpiperidin-4-yl]-7H-pyrrolo[2,3-c]pyridazin-3- |
| | yl}-5-(4-methyl-1H-1,2,3-triazol-1-yl)phenol |
| 16\hat{ } | 2-{7-[(7S)-4-azaspiro[2.5]octan-7-yl]-7H-pyrrolo[2,3-c]pyridazin-3-yl}-5-(4-methyl- |
| | 1H-1,2,3-triazol-1-yl)phenol |
| 17\hat{ } | 2-{7-[(3S,4S)-3-fluoro-2,2,6,6-tetramethylpiperidin-4-yl]-7H-pyrrolo[2,3-c]pyridazin-3- |
| | yl}-5-(5-methyl-1H-1,2,3-triazol-1-yl)phenol |
| 18\hat{ } | 2-{7-[(7S)-4-azaspiro[2.5]octan-7-yl]-7H-pyrrolo[2,3-c]pyridazin-3-yl}-5-(5-methyl- |
| | 1H-1,2,3-triazol-1-yl)phenol |
| 19\hat{ } | 5-(4-chloro-1H-1,2,3-triazol-1-yl)-2-{7-[(3S,4S)-3-fluoro-2,2,6,6-tetramethylpiperidin- |
| | 4-yl]-7H-pyrrolo[2,3-c]pyridazin-3-yl}phenol |
| 20 | 2-{7-[(1S,2S,3R,5R)-2-fluoro-8-azabicyclo[3.2.1]octan-3-yl]-7H-pyrrolo[2,3- |
| | c]pyridazin-3-yl}-5-(1H-1,2,3-triazol-1-yl)phenol and |
| | 2-{7-[(1R,2R,3S,5S)-2-fluoro-8-azabicyclo[3.2.1]octan-3-yl]-7H-pyrrolo[2,3- |
| | c]pyridazin-3-yl}-5-(1H-1,2,3-triazol-1-yl)phenol |
| 21 | 2-{7-[(1S,2R,3R,5R)-2-fluoro-1,5-dimethyl-8-azabicyclo[3.2.1]octan-3-yl]-7H- |
| | pyrrolo[2,3-c]pyridazin-3-yl}-5-(1H-1,2,3-triazol-1-yl)phenol and |
| | 2-{7-[(1R,2S,3S,5S)-2-fluoro-1,5-dimethyl-8-azabicyclo[3.2.1]octan-3-yl]-7H- |
| | pyrrolo[2,3-c]pyridazin-3-yl}-5-(1H-1,2,3-triazol-1-yl)phenol |
| 22 | 2-{7-[(4S,5R)-5-fluoro-2,2-dimethylpiperidin-4-yl]-7H-pyrrolo[2,3-c]pyridazin-3-yl}-5- |
| | (1H-1,2,3-triazol-1-yl)phenol |
| 23 | 2-{7-[(8R,9R)-9-fluoro-5-azaspiro[3.5]nonan-8-yl]-7H-pyrrolo[2,3-c]pyridazin-3-yl}-5- |
| | (1H-1,2,3-triazol-1-yl)phenol and |
| | 2-{7-[(8S,9S)-9-fluoro-5-azaspiro[3.5]nonan-8-yl]-7H-pyrrolo[2,3-c]pyridazin-3-yl}-5- |
| | (1H-1,2,3-triazol-1-yl)phenol |
| 24 | 2-[7-(4-azadispiro[2.1.2⁵.3³]decan-9-yl)-7H-pyrrolo[2,3-c]pyridazin-3-yl]-5-(1H-1,2,3- |
| | triazol-1-yl)phenol |
| 25 | 2-{7-[(8S,9R)-8-fluoro-6-azaspiro[4.5]decan-9-yl]-7H-pyrrolo[2,3-c]pyridazin-3-yl}-5- |
| | (1H-1,2,3-triazol-1-yl)phenol and |
| | 2-{7-[(8R,9S)-8-fluoro-6-azaspiro[4.5]decan-9-yl]-7H-pyrrolo[2,3-c]pyridazin-3-yl}-5- |
| | (1H-1,2,3-triazol-1-yl)phenol |
| 26 | 4-fluoro-2-{7-[(3S,4S)-3-fluoro-2,2,6,6-tetramethylpiperidin-4-yl]-7H-pyrrolo[2,3- |
| | c]pyridazin-3-yl}-5-(1H-1,2,3-triazol-1-yl)pheno, and |
| 27 | 3-fluoro-2-{7-[(3S,4S)-3-fluoro-2,2,6,6-tetramethylpiperidin-4-yl]-7H-pyrrolo[2,3- |
| | c]pyridazin-3-yl}-5-(1H-1,2,3-triazol-1-yl)phenol; |

wherein the form of the compound is selected from the group consisting of a salt, racemate, enantiomer, diastereomer, stereoisomer, and tautomer form thereof.

Another aspect of the compound of Formula (I) or a form thereof is a compound salt selected from the group consisting of:

| Cpd | Name |
|---|---|
| 1 | 2-[7-(2,2,6,6-tetramethylpiperidin-4-yl)-7H-pyrrolo[2,3-c]pyridazin-3-yl]-5-(1H-1,2,3- |
| | triazol-1-yl)phenol dihydrochloride |
| 2 | 2-{7-[(3S,4S)-3-fluoro-2,2,6,6-tetramethylpiperidin-4-yl]-7H-pyrrolo[2,3-c]pyridazin-3- |
| | yl}-5-(1H-1,2,3-triazol-1-yl)phenol dihydrochloride and |
| | 2-{7-[(3R,4R)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol dihydrochloride |
| 3 | 2-{7-[(3R,4S)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol dihydrochloride and |
| | 2-{7-[(3S,4R)-3-fluoro-2,2,6,6-tetramethylpiperidin-4-yl]-7H-pyrrolo[2,3-c]pyridazin- |
| | 3-yl}-5-(1H-1,2,3-triazol-1-yl)phenol dihydrochloride |
| 4 | 2-[7-(4-azaspiro[2.5]octan-7-yl)-7H-pyrrolo[2,3-c]pyridazin-3-yl]-5-(1H-1,2,3-triazol- |
| | 1-yl)phenol dihydrochloride |
| 5 | 2-{7-[(4RS)-2,2-dimethylpiperidin-4-yl]-7H-pyrrolo[2,3-c]pyridazin-3-yl}-5-(1H-1,2,3- |
| | triazol-1-yl)phenol hydrochloride |
| 6 | 5-(1H-1,2,3-triazol-1-yl)-2-{7-[(4RS)-1,2,2-trimethylpiperidin-4-yl]-7H-pyrrolo[2,3- |
| | c]pyridazin-3-yl}phenol hydrochloride |
| 7 | 2-[7-(4-methyl-4-azaspiro[2.5]octan-7-yl)-7H-pyrrolo[2,3-c]pyridazin-3-yl]-5-(1H- |
| | 1,2,3-triazol-1-yl)phenol hydrochloride |
| 8 | 2-{7-[(3S,4S)-3-fluoro-2,2,6,6-tetramethylpiperidin-4-yl]-7H-pyrrolo[2,3-c]pyridazin-3- |
| | yl}-5-(1H-1,2,3-triazol-1-yl)phenol dihydrochloride |
| 9 | 2-[7-(4-azaspiro[2.5]octan-7-yl)-6-methyl-7H-pyrrolo[2,3-c]pyridazin-3-yl]-5-(1H- |
| | 1,2,3-triazol-1-yl)phenol diformate |
| 10 | 2-{7-[(3S,4S)-3-fluoro-2,2,6,6-tetramethylpiperidin-4-yl]-6-methyl-7H-pyrrolo[2,3- |
| | c]pyridazin-3-yl}-5-(1H-1,2,3-triazol-1-yl)phenol diformate |
| 12 | 2-{7-[(4S,5R)-5-fluoro-2,2-dimethylpiperidin-4-yl]-7H-pyrrolo[2,3-c]pyridazin-3-yl}-5- |
| | (1H-1,2,3-triazol-1-yl)phenol hydrochloride and |
| | 2-{7-[(4R,5S)-5-fluoro-2,2-dimethylpiperidin-4-yl]-7H-pyrrolo[2,3-c]pyridazin-3-yl}-5- |
| | (1H-1,2,3-triazol-1-yl)phenol hydrochloride |
| 13 | 2-{7-[(7S)-4-azaspiro[2.5]octan-7-yl]-7H-pyrrolo[2,3-c]pyridazin-3-yl}-5-(1H-1,2,3- |
| | triazol-1-yl)phenol trifluoroacetate |
| 14 | 2-{7-[(7S)-4-azaspiro[2.5]octan-7-yl]-7H-pyrrolo[2,3-c]pyridazin-3-yl}-5-(1H-1,2,3- |
| | triazol-1-yl)pyridine-3-ol trifluoroacetate |
| 15 | 2-{7-[(3S,4S)-3-fluoro-2,2,6,6-tetramethylpiperidin-4-yl]-7H-pyrrolo[2,3-c]pyridazin-3- |
| | yl}-5-(4-methyl-1H-1,2,3-triazol-1-yl)phenol formate |
| 16 | 2-{7-[(7S)-4-azaspiro[2.5]octan-7-yl]-7H-pyrrolo[2,3-c]pyridazin-3-yl}-5-(4-methyl- |
| | 1H-1,2,3-triazol-1-yl)phenol formate |
| 17 | 2-{7-[(3S,4S)-3-fluoro-2,2,6,6-tetramethylpiperidin-4-yl]-7H-pyrrolo[2,3-c]pyridazin-3- |
| | yl}-5-(5-methyl-1H-1,2,3-triazol-1-yl)phenol formate |
| 18 | 2-{7-[(7S)-4-azaspiro[2.5]octan-7-yl]-7H-pyrrolo[2,3-c]pyridazin-3-yl}-5-(5-methyl- |
| | 1H-1,2,3-triazol-1-yl)phenol formate formate, and |
| 19 | 5-(4-chloro-1H-1,2,3-triazol-1-yl)-2-{7-[(3S,4S)-3-fluoro-2,2,6,6-tetramethylpiperidin- |
| | 4-yl]-7H-pyrrolo[2,3-c]pyridazin-3-yl}phenol formate; |

wherein the form of the compound salt is selected from the group consisting of a racemate, enantiomer, diastereomer, stereoisomer, and tautomer form thereof.

An aspect of the present description includes a method of use of a compound of Formula (I) or a form thereof for treating or ameliorating HD in a subject in need thereof, comprising administering an effective amount of the compound of Formula (I) or a form thereof to the subject.

Another aspect of the present description includes a method of use of the compound salt of Formula (I) or a form thereof for treating or ameliorating HD in a subject in need thereof, comprising administering an effective amount of the compound salt of Formula (I) or a form thereof to the subject.

An aspect of the present description includes a use of the compound of Formula (I) or a form thereof for treating or ameliorating HD in a subject in need thereof, comprising administering an effective amount of the compound of Formula (I) or a form thereof to the subject.

Another aspect of the present description includes a use of the compound salt of Formula (I) or a form thereof for treating or ameliorating HD in a subject in need thereof, comprising administering an effective amount of the compound salt of Formula (I) or a form thereof to the subject.

### Chemical Definitions

The chemical terms used above and throughout the description herein, unless specifically defined otherwise, shall be understood by one of ordinary skill in the art to have the following indicated meanings.

As used herein, the term “C₁₋₄alkyl” generally refers to saturated hydrocarbon radicals having from one to four carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl and the like. In certain aspects, C₁₋₄alkyl includes, but is not limited to C₁₋₄alkyl and the like. A C₁₋₄alkyl radical is optionally substituted with substituent species as described herein where allowed by available valences.

As used herein, the term “C₂₋₄alkenyl” generally refers to partially unsaturated hydrocarbon radicals having from two to four carbon atoms in a straight or branched chain configuration and one or more carbon-carbon double bonds therein, including, but not limited to, ethenyl (also referred to as vinyl), allyl, and propenyl, and butenyl. In certain aspects, C₂₋₄alkenyl includes, but is not limited to, C₂₋₃alkenyl and C₂₋₄alkenyl. A C₂₋₄alkenyl radical is optionally substituted with substituent species as described herein where allowed by available valences.

As used herein, the term “C₂₋₄alkynyl” generally refers to partially unsaturated hydrocarbon radicals having from two to four carbon atoms in a straight or branched chain configuration and one or more carbon-carbon triple bonds therein, including, but not limited to, ethynyl, propynyl, and butynyl. In certain aspects, C₂₋₄alkynyl includes, but is not limited to, C₂₋₃alkynyl and C₂₋₄alkynyl. A C₂₋₄alkynyl radical is optionally substituted with substituent species as described herein where allowed by available valences.

As used herein, the term “C₁₋₄alkoxy” generally refers to saturated hydrocarbon radicals having from one to four carbon atoms in a straight or branched chain configuration of the formula: —O—C₁₋₄alkyl, including, but not limited to, methoxy, ethoxy, n-propoxy, isopropoxy, n-butoxy, isobutoxy, sec-butoxy, tert-butoxy and the like. In certain aspects, C₁₋₄alkoxy includes, but is not limited to C₁₋₄alkoxy and the like. A C₁₋₄alkoxy radical is optionally substituted with substituent species as described herein where allowed by available valences.

As used herein, the term “C₃₋₁₀cycloalkyl” generally refers to a saturated or partially unsaturated monocyclic, bicyclic or polycyclic hydrocarbon radical, including, but not limited to, cyclopropyl, cyclobutyl, cyclopentyl, cyclohexyl, cyclohexenyl, cycloheptyl, cyclooctyl, 1H-indanyl, indenyl, tetrahydro-naphthalenyl and the like. In certain aspects, C₃₋₁₀cycloalkyl includes, but is not limited to C₃₋₈cycloalkyl, C₅₋₈cycloalkyl, C₃₋₁₀cycloalkyl and the like. A C₃₋₁₀cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thienyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothienyl, benzoimidazolyl, 1,3-benzothiazolyl, 1,3-benzoxazolyl, purinyl, quinolinyl, isoquinolinyl, quinazolinyl, quinoxalinyl, 1,3-diazinyl, 1,2-diazinyl, 1,2-diazolyl, 1,4-diazanaphthalenyl, acridinyl, furo[3,2-b]pyridinyl, furo[3,2-c]pyridinyl, furo[2,3-c]pyridinyl, 6H-thieno[2,3-b]pyrrolyl, thieno[3,2-c]pyridinyl, thieno[2,3-d]pyrimidinyl, 1H-pyrrolo[2,3-b]pyridinyl, 1H-pyrrolo[2,3-c]pyridinyl, 1H-pyrrolo[3,2-b]pyridinyl, pyrrolo[1,2-a]pyrazinyl, pyrrolo[1,2-b]pyridazinyl, pyrazolo[1,5-a]pyridinyl, pyrazolo[1,5-a]pyrazinyl, imidazo[1,2-a]pyridinyl, 3H-imidazo[4,5-b]pyridinyl, imidazo[1,2-a]pyrimidinyl, imidazo[1,2-c]pyrimidinyl, imidazo[1,2-b]pyridazinyl, imidazo[1,2-a]pyrazinyl, imidazo[2,1-b][1,3]thiazolyl, imidazo[2,1-b][1,3,4]thiadiazolyl, [1,2,4]triazolo[1,5-a]pyridinyl, [1,2,4]triazolo[4,3-a]pyridinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thienyl may also be referred to as thiophenyl, pyridinyl may also be referred to as pyridyl, benzothienyl may also be referred to as benzothiophenyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl. 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxctanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, thiopyranyl, 1,3-dioxanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl, 2,3-dihydro-1,4-benzodioxinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, (3aR,6aR)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, (3aR,6aR)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS,7aS)-octahydro-6H-pyrrolo[3,4-b]pyridinyl, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-octahydropyrrolo[1,2-a]pyrazin-(1H)-yl, (8aR)-octahydropyrrolo[1,2-a]pyrazin-(1H)-yl, hexahydropyrrolo[1,2-a]pyrazin-(2H)-one, octahydro-2H-pyrido[1,2-a]pyrazinyl, 3-azabicyclo[3.1.0]hexyl, (1R,5S)-3-azabicyclo[3.1.0]hexyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, azaspiro[3.3]heptyl, 2,6-diazaspiro[3.3]heptyl, 2,7-diazaspiro[3.5]nonyl, 5,8-diazaspiro[3.5]nonyl, 2,7-diazaspiro[4.4]nonyl, 6,9-diazaspiro[4.5]decyl and the like. A heterocyclyl radical is optionally substituted on a carbon or nitrogen atom ring member with substituent species as described herein where allowed by available valences.

In certain aspects, the nomenclature for a heterocyclyl radical may differ, such as in non-limiting examples where 1,3-benzodioxolyl may also be referred to as benzo[d][1,3]dioxolyl and 2,3-dihydro-1,4-benzodioxinyl may also be referred to as 2,3-dihydrobenzo[b][1,4]dioxinyl.

As used herein, the term “deutero-C₁₋₄alkyl,” refers to a radical of the formula: —C₁₋₄alkyl-deutero, wherein C₁₋₄alkyl is partially or completely substituted with one or more deuterium atoms where allowed by available valences.

As used herein, the term “C₁₋₄alkoxy-C₁₋₄alkyl” refers to a radical of the formula: —C₁₋₄alkyl-O—C₁₋₄alkyl.

As used herein, the term “C₁₋₄alkyl-amino” refers to a radical of the formula: —NH—C₁₋₄alkyl.

As used herein, the term “(C₁₋₄alkyl)₂-amino” refers to a radical of the formula: —N(C₁₋₄alkyl)₂.

As used herein, the term “C₁₋₄alkyl-thio” refers to a radical of the formula: —S—C₁₋₄alkyl.

As used herein, the term “amino-C₁₋₄alkyl” refers to a radical of the formula: —C₁₋₄alkyl-NH₂.

As used herein, the term “halo” or “halogen” generally refers to a halogen atom radical, including fluoro, chloro, bromo and iodo.

As used herein, the term “halo-C₁₋₄alkoxy” refers to a radical of the formula: —O—C₁₋₄alkyl-halo, wherein C₁₋₄alkyl is partially or completely substituted with one or more halogen atoms where allowed by available valences.

As used herein, the term “halo-C₁₋₄alkyl” refers to a radical of the formula: —C₁₋₄alkyl-halo, wherein C₁₋₄alkyl is partially or completely substituted with one or more halogen atoms where allowed by available valences.

As used herein, the term “hydroxy” refers to a radical of the formula:—OH.

As used herein, the term “hydroxy-C₁₋₄alkyl” refers to a radical of the formula: —C₁₋₄alkyl-OH, wherein C₁₋₄alkyl is partially or completely substituted with one or more hydroxy radicals where allowed by available valences.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence. Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₄cycloalkyl, C₃₋₁₄cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₄cycloalkyl, aryl, heteroaryl and heterocyclyl ring systems when each are present either alone or as a substituent.

As used herein, the term “optionally substituted” means optional substitution with the specified substituent variables, groups, radicals or moieties.

### Compound Forms

As used herein, the term “form” means a compound of Formula (I) having a form selected from the group consisting of a free acid, free base, salt, hydrate, solvate, racemate, enantiomer, diastereomer, stereoisomer, and tautomer form thereof.

In certain aspects described herein, the form of the compound of Formula (I) is a free acid, free base or salt thereof.

In certain aspects described herein, the form of the compound of Formula (I) is a salt thereof.

In certain aspects described herein, the form of the compound of Formula (I) is a stereoisomer, racemate, enantiomer or diastereomer thereof.

In certain aspects described herein, the form of the compound of Formula (I) is a tautomer thereof.

In certain aspects described herein, the form of the compound of Formula (I) is an isotopologue thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride, bromide or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66 (1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R/S) or as substantially pure enantiomers and diastereomers. The compounds may also exist as substantially pure (R) or(S) enantiomers (when one chiral center is present). In one particular aspect, the compounds described herein are(S) isomers and may exist as enantiomerically pure compositions substantially comprising only the(S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “chiral” refers to a carbon atom bonded to four nonidentical substituents. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stercochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511).

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I) or a form thereof is a substantially pure(S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

The use of the terms “salt”, “solvate”, and the like, is intended to equally apply to the salt, solvate, enantiomers, stereoisomers, or tautomers, of the instant compounds.

The term “isotopologue” refers to isotopically-enriched compounds described herein which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, ³⁵Cl and ³⁶Cl, respectively, each of which are also within the scope of this description.

### Compound Uses

An aspect of the present description relates to a method of use of a compound of Formula (I) or a form thereof for treating or ameliorating HD in a subject in need thereof, comprising administering an effective amount of the compound or a form thereof to the subject.

Another aspect of the present description relates to use of the compound of Formula (I) or a form thereof for treating or ameliorating HD in a subject in need thereof.

Another aspect of the present description relates to use of the compound of Formula (I) or a form thereof having activity toward HD.

An aspect of the present description relates to use of the compound of Formula (I) or a form thereof in a combination therapy to provide additive or synergistic activity, thus enabling the development of a combination product for treating or ameliorating HD.

In addition to monotherapeutic use, the instant compounds are useful in a combination therapy with current standard of agents, having additive or synergistic activity with one or more known agents.

A combination therapy comprising compounds described herein in combination with one or more known drugs may be used to treat HD regardless of whether HD is responsive to the known drug.

Certain aspects of the present description include the use of a compound of Formula (I) or a form thereof in a combination therapy for treating or ameliorating HD in a subject in need thereof, comprising administering an effective amount of the compound of Formula (I) or a form thereof and an effective amount of one or more agent(s).

Certain particular aspects of the present description include the use of a compound of Formula (I) or a form thereof in a combination therapy for treating or ameliorating HD in a subject in need thereof, comprising administering an effective amount of the compound of Formula (I) or a form thereof and an effective amount of one or more agent(s).

In an aspect of a use or method provided herein, compounds of Formula (I) or a form thereof used in combination with one or more additional agents can be administered to a subject or contacted with a subject or patient cell(s) prior to, concurrently with, or subsequent to administering to the subject or patient or contacting the cell with an additional agent(s). A compound(s) of Formula (I) or a form thereof and an additional agent(s) can be administered to a subject or contacted with a cell in single composition or different compositions. In a specific aspect, a compound(s) of Formula (I) or a form thereof is used in combination with gene therapy to inhibit HTT expression (using, e.g., viral delivery vectors) or the administration of another small molecule HTT inhibitor. In another specific aspect, a compound(s) of Formula (I) or a form thereof are used in combination with cell replacement using differentiated non-mutant HTT stem cells. In another specific aspect, a compound(s) of Formula (I) or a form thereof are used in combination with cell replacement using differentiated HTT stem cells.

In one aspect, provided herein is the use of compounds of Formula (I) or a form thereof in combination with supportive standard of care therapies, including palliative care.

An aspect of the present description includes the use of a compound of Formula (I) or a form thereof in the preparation of a kit comprising the compound of Formula (I) or a form thereof and instructions for administering an effective amount of the compound of Formula (I) or a form thereof and an effective amount of one or more agent(s) in a combination therapy for treating or ameliorating HD in a subject in need thereof.

Accordingly, the present description relates to use of a compound of Formula (I) or a form thereof for treating or ameliorating HD. In accordance with the use of the present description, compounds that are useful in selectively treating or ameliorating HD, have been identified and use of these compounds for treating or ameliorating HD has been provided.

Another aspect of the use of the present description relates to use of a compound of Formula (I) or a form thereof for treating or ameliorating HD in a subject in need thereof, comprising administering an effective amount of the compound of Formula (I) or a form thereof to the subject.

Another aspect of the use of the present description relates to a method of use of a compound of Formula (I) or a form thereof for treating or ameliorating HD in a subject in need thereof, comprising administering an effective amount of the compound to the subject.

Another aspect of the use of the present description relates to a method of use of a compound of Formula (I) or a form thereof for treating or ameliorating HD in a subject in need thereof, comprising administering an effective amount of the compound to the subject.

Another aspect of the use of the present description relates to use of a compound of Formula (I) or a form thereof in the manufacture of a medicament for treating or ameliorating HD in a subject in need thereof, comprising administering an effective amount of the medicament to the subject.

Another aspect of the use of the present description relates to use of a compound of Formula (I) or a form thereof in the preparation of a kit comprising the compound of Formula (I) or a form thereof and instructions for administering the compound for treating or ameliorating HD in a subject in need thereof.

In one respect, for each of such aspects, the subject is treatment naive. In another respect, for each of such aspects, the subject is not treatment naive.

As used herein, the term “treating” refers to: (i) preventing a disease, disorder or condition from occurring in a subject that may be predisposed to the disease, disorder and/or condition but has not yet been diagnosed as having the disease, disorder and/or condition; (ii) inhibiting a disease, disorder or condition, i.e., arresting the development thereof; and/or (iii) relieving a disease, disorder or condition, i.e., causing regression of the disease, disorder and/or condition.

As used herein, the term “subject” refers to an animal or any living organism having sensation and the power of voluntary movement, and which requires oxygen and organic food. Nonlimiting examples include members of the human, primate, equine, porcine, bovine, murine, rattus, canine and feline specie. In certain aspects, the subject is a mammal or a warm-blooded vertebrate animal. In other aspects, the subject is a human. As used herein, the term “patient” may be used interchangeably with “subject” and “human”.

As used herein, the terms “effective amount” or “therapeutically effective amount” mean an amount of compound of Formula (I) or a form, composition or medicament thereof that achieves a target plasma concentration that is effective in treating or ameliorating HD as described herein and thus producing the desired therapeutic, ameliorative, inhibitory or preventative effect in a subject in need thereof. In one aspect, the effective amount may be the amount required to treat HD in a subject or patient, more specifically, in a human.

In another aspect, the concentration-biological effect relationships observed with regard to a compound of Formula (I) or a form thereof indicate a target plasma concentration ranging from approximately 0.001 μg/mL to approximately 50 μg/mL, from approximately 0.01 μg/mL to approximately 20 μg/mL, from approximately 0.05 μg/mL to approximately 10 μg/mL, or from approximately 0.1 μg/mL to approximately 5 μg/mL. To achieve such plasma concentrations, the compounds described herein may be administered at doses that vary, such as, for example, without limitation, from 0.1 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12 h”), thrice (once in approximately an 8 hour period; i.e., “t.i.d.” or “q.8 h”), or four times (once in approximately a 6 hour period; i.e., “q.d.s.”, “q.i.d.” or “q.6 h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intravenous (bolus and infusion), intracerebral, and pulmonary routes of administration.

In another aspect, the dose administered may be adjusted based upon a dosage form described herein formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

In one aspect, provided herein are methods for modulating the amount of HTT (huntingtin protein), comprising contacting a human cell with a compound of Formula (I) or a form thereof. In a specific aspect, provided herein are methods for modulating the amount of HTT, comprising contacting a human cell with a compound of Formula (I) or a form thereof that modulates the expression of HTT. The human cell can be contacted with a compound of Formula (I) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of HTT expression and/or function. In another aspect, the human cell is from a human with HD. In another aspect, the human cell is in a human with HD. In one aspect, the compound is a form of the compound of Formula (I).

In a specific aspect, provided herein is a method for enhancing the inhibition of mutant HTT transcribed from the Htt gene, comprising contacting a human cell with a compound of Formula (I) or a form thereof. The human cell can be contacted with a compound of Formula (I) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of wild-type “normal” HTT expression and/or function. In another aspect, the human cell is from a human with HD. In another aspect, the human cell is in a human with HD. In one aspect, the compound is a form of the compound of Formula (I).

In another aspect, provided herein is a method for modulating the inhibition of mutant HTT transcribed from the Htt gene, comprising administering to a non-human animal model for HD a compound of Formula (I) or a form thereof. In a specific aspect, provided herein is a method for modulating the inhibition of mutant HTT transcribed from the Htt gene, comprising administering to a non-human animal model for HD a compound of Formula (I) or a form thereof. In a specific aspect, the compound is a form of the compound of Formula (I).

In another aspect, provided herein is a method for decreasing the amount of mutant HTT, comprising contacting a human cell with a compound of Formula (I) or a form thereof. In a specific aspect, provided herein is a method for decreasing the amount of mutant HTT, comprising contacting a human cell with a compound of Formula (I) that inhibits the transcription of mutant HTT (huntingtin mRNA) from the Htt gene. In another specific aspect, provided herein is a method for decreasing the amount of HTT, comprising contacting a human cell with a compound of Formula (I) that inhibits the expression of mutant HTT transcribed from the Htt gene. The human cell can be contacted with a compound of Formula (I) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of HTT expression and/or function. In another aspect, the human cell is from a human with HD. In another aspect, the human cell is in a human with HD. In one aspect, the compound is a form of the compound of Formula (I).

In certain aspects, treating or ameliorating HD with a compound of Formula (I) or a form thereof (alone or in combination with an additional agent) has a therapeutic effect and/or beneficial effect. In a specific aspect, treating HD with a compound of Formula (I) or a form thereof (alone or in combination with an additional agent) results in one, two or more of the following effects: (i) reduces or ameliorates the severity of HD; (ii) delays onset of HD; (iii) inhibits the progression of HD; (iv) reduces hospitalization of a subject; (v) reduces hospitalization length for a subject; (vi) increases the survival of a subject; (vii) improves the quality of life for a subject; (viii) reduces the number of symptoms associated with HD; (ix) reduces or ameliorates the severity of a symptom(s) associated with HD; (x) reduces the duration of a symptom associated with HD; (xi) prevents the recurrence of a symptom associated with HD; (xii) inhibits the development or onset of a symptom of HD; and/or (xiii) inhibits of the progression of a symptom associated with HD.

### Metabolites

Also included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

### Pharmaceutical Compositions

Aspects of the present description include the use of a compound of Formula (I) or a form thereof in a pharmaceutical composition for treating or ameliorating HD in a subject in need thereof, comprising administering an effective amount of the compound of Formula (I) or a form thereof in admixture with one or more pharmaceutically acceptable excipient(s).

An aspect of the present description includes the use of a pharmaceutical composition of the compound of Formula (I) or a form thereof in the preparation of a kit comprising the pharmaceutical composition of the compound of Formula (I) or a form thereof and instructions for administering the compound for treating or ameliorating HD in a subject in need thereof.

As used herein, the term “composition” means a product comprising the specified ingredients in the specified amounts, as well as any product which results, directly or indirectly, from combination of the specified ingredients in the specified amounts.

The pharmaceutical composition may be formulated to achieve a physiologically compatible pH, ranging from about pH 3 to about pH 11. In certain aspects, the pharmaceutical composition is formulated to achieve a pH of from about pH 3 to about pH 7. In other aspects, the pharmaceutical composition is formulated to achieve a pH of from about pH 5 to about pH 8.

The term “pharmaceutically acceptable excipient” refers to an excipient for administration of a pharmaceutical agent, such as the compounds described herein. The term refers to any pharmaceutical excipient that may be administered without undue toxicity. Pharmaceutically acceptable excipients may be determined in part by the particular composition being administered, as well as by the particular mode of administration and/or dosage form. Nonlimiting examples of pharmaceutically acceptable excipients include carriers, solvents, stabilizers, adjuvants, diluents, etc. Accordingly, there exists a wide variety of suitable formulations of pharmaceutical compositions for the instant compounds described herein (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive antibodies. Other exemplary excipients include antioxidants such as ascorbic acid; chelating agents such as EDTA; carbohydrates such as dextrin, hydroxyalkylcellulose, hydroxyalkylmethylcellulose (e.g., hydroxypropylmethylcellulose, also known as HPMC), stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended use described herein. Suitable formulations for oral administration include solids, liquid solutions, emulsions and suspensions, while suitable inhalable formulations for pulmonary administration include liquids and powders. Alternative formulations include syrups, creams, ointments, tablets, and lyophilized solids which can be reconstituted with a physiologically compatible solvent prior to administration.

When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents, in order to provide a palatable preparation.

Pharmaceutically acceptable excipients suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid, or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate, or kaolin, or as soft gelatin capsules wherein the active ingredient is mixed with non-aqueous or oil medium, such as glycerin, propylene glycol, polyethylene glycol, peanut oil, liquid paraffin, or olive oil.

In other aspects, pharmaceutical compositions described herein may be formulated as suspensions comprising a compound of Formula (I) or a form thereof in admixture with one or more pharmaceutically acceptable excipient(s) suitable for the manufacture of a suspension. In yet other aspects, pharmaceutical compositions described herein may be formulated as dispersible powders and granules suitable for preparation of a suspension by the addition of one or more excipient(s).

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions described herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions described herein may be in the form of a sterile injectable preparation, such as a sterile injectable aqueous emulsion or oleaginous suspension. Such emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propanediol. The sterile injectable preparation may also be prepared as a lyophilized powder. Among the acceptable vehicles and solvents that may be employed are water, Ringer's solution and isotonic sodium chloride solution. In addition, sterile fixed oils may be employed as a solvent or suspending medium. For this purpose any bland fixed oil may be employed including synthetic mono- or di-glycerides. In addition, fatty acids such as oleic acid may likewise be used in the preparation of injectables.

The compounds described herein may be substantially insoluble in water and sparingly soluble in most pharmaceutically acceptable protic solvents and vegetable oils, but generally soluble in medium-chain fatty acids (e.g., caprylic and capric acids) or triglycerides and in propylene glycol esters of medium-chain fatty acids. Thus, contemplated in the description are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In certain aspects, the compound described herein is formulated for oral administration in a lipid-based composition suitable for low solubility compounds. Lipid-based formulations can generally enhance the oral bioavailability of such compounds. As such, pharmaceutical compositions described herein may comprise a effective amount of a compound of Formula (I) or a form thereof, together with at least one pharmaceutically acceptable excipient selected from medium chain fatty acids or propylene glycol esters thereof (e.g., propylene glycol esters of edible fatty acids such as caprylic and capric fatty acids) and pharmaceutically acceptable surfactants, such as polysorbate 20 or 80 (also referred to as Tween® 20 or Tween® 80, respectively) or polyoxyl 40 hydrogenated castor oil.

In other aspects, the bioavailability of low solubility compounds may be enhanced using particle size optimization techniques including the preparation of nanoparticles or nanosuspensions using techniques known to those skilled in the art. The compound forms present in such preparations include amorphous, partially amorphous, partially crystalline or crystalline forms.

In alternative aspects, the pharmaceutical composition may further comprise one or more aqueous solubility enhancer(s), such as a cyclodextrin. Nonlimiting examples of cyclodextrin include hydroxypropyl, hydroxyethyl, glucosyl, maltosyl and maltotriosyl derivatives of α-, β-, and γ-cyclodextrin, and hydroxypropyl-β-cyclodextrin (HPBC). In certain aspects, the pharmaceutical composition further comprises HPBC in a range of from about 0.1% to about 20%, from about 1% to about 15%, or from about 2.5% to about 10%. The amount of solubility enhancer employed may depend on the amount of the compound in the composition.

### Preparation of Compounds

### General Synthetic Methods

As disclosed herein, general methods for preparing the compounds of Formula (I) or a form thereof as described herein are available via standard, well-known synthetic methodology. Many of the starting materials are commercially available or, when not available, can be prepared using the routes described below using techniques known to those skilled in the art. The synthetic schemes provided herein comprise multiple reaction steps, each of which is intended to stand on its own and can be carried out with or without any preceding or succeeding step(s). In other words, each of the individual reaction steps of the synthetic schemes provided herein in isolation is contemplated.

Compounds of Formula (I) may be prepared as described in Scheme 1 below.

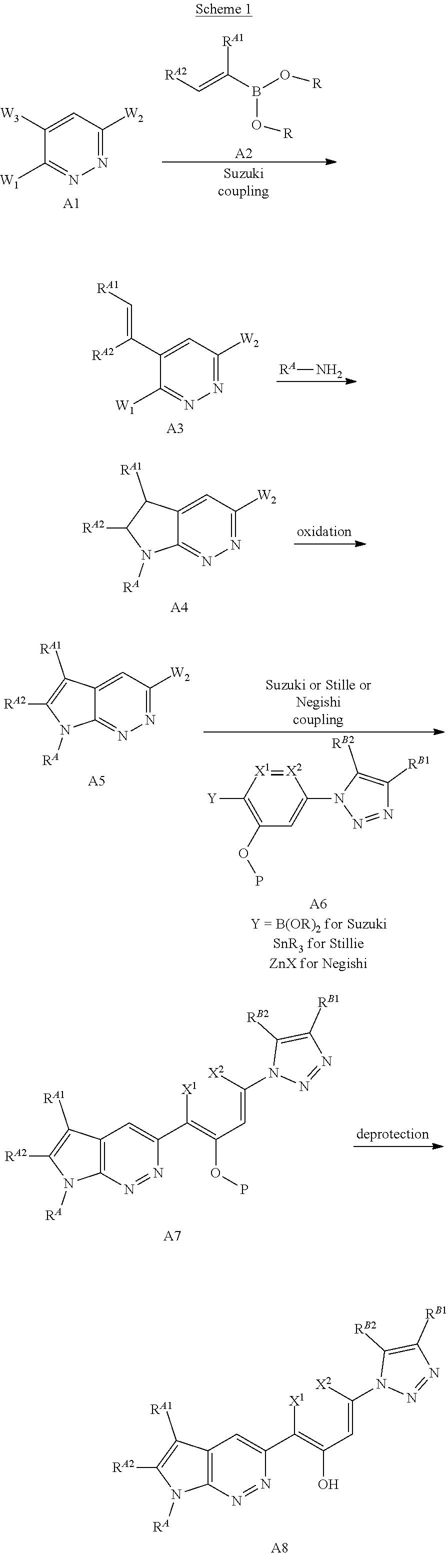

Compound A1 (where W₁, W₂ and W₃ are independently bromo, chloro and the like) is converted to Compound A3 by a Suzuki coupling with a pinacol boronic ester (or boronic acid) A2 in the presence of a catalyst (such as Pd(dppf)Cl₂ and the like) and base (such as aqueous K₂CO₃ and the like) in a suitable solvent (such as 1,4-dioxane and the like). Compound A3 is converted to Compound A4 by heating with a primary amine (R^{A}NH₂) in a suitable solvent (such as acetonitrile and the like) in the presence of a base (such as N,N-Diisopropylethylamine and the like). Compound A4 is converted to Compound A5 by treating with a suitable oxidizing agent (such as manganese dioxide and the like) in a suitable solvent (such as toluene and the like). Compound A5 is converted to Compound A7 by a Suzuki coupling with a coupling partner A6 (where Y is boronic acid or boronic ester and P is a suitable protecting group) in the presence of a catalyst (such as Pd(dppf)Cl₂ and the like) and base (such as aqueous K₂CO₃ and the like) in a suitable solvent (such as 1,4-dioxane and the like). Alternatively, Compound A5 is converted to Compound A7 by a Stille coupling with a coupling partner A6 (where Y is stannane) in the presence of a catalyst (such as Pd₂(dba)₃ and the like), a ligand (such as X-Phos and the like) and a base (such as CsF and the like) in a suitable solvent (such as 1,4-dioxane and the like). Alternatively, Compound A5 is converted to Compound A7 by a Negishi coupling with a coupling partner A6 (where Y is zinc halide) in the presence of a catalyst (such as Pd(PPh₃)₄ and the like), in a suitable solvent (such as THF and the like). Compound A7 is converted to Compound A8 upon treatment with conditions appropriate to the removal of the protecting groups (such as HCl in dioxane for a MOM protecting group) in a suitable solvent (such as dioxane and the like).

Alternatively, compounds of Formula (I) may be prepared as described in Scheme 2 below.

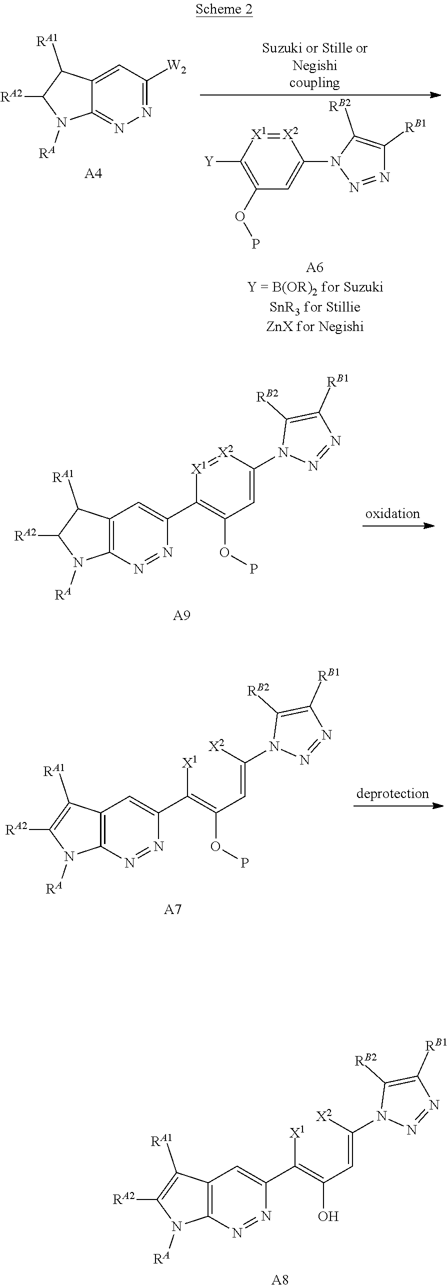

Compound A4 is converted to Compound A9 by a Suzuki coupling with a coupling partner A6 (where Y is boronic acid or boronic ester) in the presence of a catalyst (such as Pd(dppf)Cl₂ and the like) and base (such as aqueous K₂CO₃ and the like) in a suitable solvent (such as 1,4-dioxane and the like). Alternatively, Compound A4 is converted to Compound A9 by a Stille coupling with a coupling partner A6 (where Y is stannane) in the presence of a catalyst (such as Pd₂(dba)₃ and the like), a ligand (such as X-Phos and the like) and a base (such as CsF and the like) in a suitable solvent (such as 1,4-dioxane and the like). Alternatively, Compound A4 is converted to Compound A9 by a Negishi coupling with a coupling partner A6 (where Y is zinc halide and P is a suitable protecting group) in the presence of a catalyst (such as Pd(PPh₃)₄ and the like), in a suitable solvent (such as THF and the like). Compound A9 is converted to compound A7 by treating with a suitable oxidizing agent (such as manganese dioxide and the like) in a suitable solvent (such as toluene and the like). Compound A7 is converted to Compound A8 upon treatment with conditions appropriate to the removal of the protecting groups (such as HCl in dioxane for a MOM protecting group) in a suitable solvent (such as dioxane and the like).

### Specific Synthetic Examples

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

### Compound Examples

As used above, and throughout the present description, the following abbreviations, unless otherwise indicated, shall be understood to have the following meanings:

| Abbreviation | Meaning |
|---|---|
| Δ | heating (chemistry) or deletion (biology) |
| AcOH or HOAc | acetic acid |
| Ac₂O | acetic anhydride |
| Ar | argon |
| ACN or CH₃CN or | acetonitrile |
| MeCN | |
| atm | atmosphere(s) |
| aq. | aqueous |
| Boc | tert-butoxy-carbonyl |
| Boc₂O | di-tert-butyl dicarbonate |
| nBuLi | n-butyl lithium |
| BuOH | n-butanol |
| ° C. | degrees Centigrade |
| Cbz | carboxybenzyl or benzyloxycarbonyl |
| Celite ® or Celite | diatomaceous earth |
| d/h/hr/hrs/min/s | day(d)/hour(h, hr or hrs)/minute(min)/second(s) |
| DCM or CH₂Cl₂ | dichloromethane |
| DIEA or DIPEA | N,N-diisopropylethylamine |
| DMF | dimethylformamide |
| DMSO | dimethylsulfoxide |
| Et₃N | triethylamine |
| EA or EtOAc | ethyl acetate |
| EtOH | ethanol |
| Et₂O | diethyl ether |
| H₂ | hydrogen |
| HBr | hydrobromic acid |
| HCl | hydrochloric acid |
| H₂SO₄ | sulfuric acid |
| HCOOH | formic acid |
| K₂CO₃ | potassium carbonate |
| KOAc | potassium acetate |
| KOtBu | potassium t-butoxide |
| KOH | potassium hydroxide |
| LC/MS, LCMS or | liquid chromatographic mass spectroscopy |
| LC-MS | |
| LiOH | lithium hydroxide |
| MeOH | methanol |
| MgSO₄ | magnesium sulfate |
| MOM | methoxy methyl |
| MS | mass spectroscopy |
| NH₄Cl | ammonium chloride |
| NH₄OH | ammonium hydroxide or aqueous ammonia |
| Na₂CO₃ | sodium carbonate |
| NaHCO₃ | sodium bicarbonate |
| NaOH | sodium hydroxide |
| Na₂SO₄ | sodium sulfate |
| N₂ | nitrogen |
| NH₄Cl | ammonium chloride |
| NMR | nuclear magnetic resonance |
| PE or pet. ether | petroleum ether |
| PhMe | toluene |
| Psi | pounds per square inch pressure |
| rt | room temperature |
| RuPhos | 2-dicyclohexylphosphino-2′,6′-diisopropoxybiphenyl |
| sat. | saturated |
| SOCl₂ | thionyl chloride |
| SO₂Cl₂ | sulfuryl chloride |
| TEA, Et₃N or NEt₃ | triethylamine |
| TFA | trifluoroacetic acid |
| THF | tetrahydrofuran |
| TLC | thin layer chromatography |
| TMS | trimethylsilane |
| TMSCl | trimethylchlorosilane or trimethylsilyl chloride |
| t-Bu | tert-butyl |
| UPLC | ultra performance liquid chromatography |

### Preparation of Starting Materials

### Preparation of 1-(3-(Methoxymethoxy)-4-(4,4,5,5-tetramethyl-1,3,2-dioxaborolan-2-yl)phenyl)-1H-1,2,3-triazole

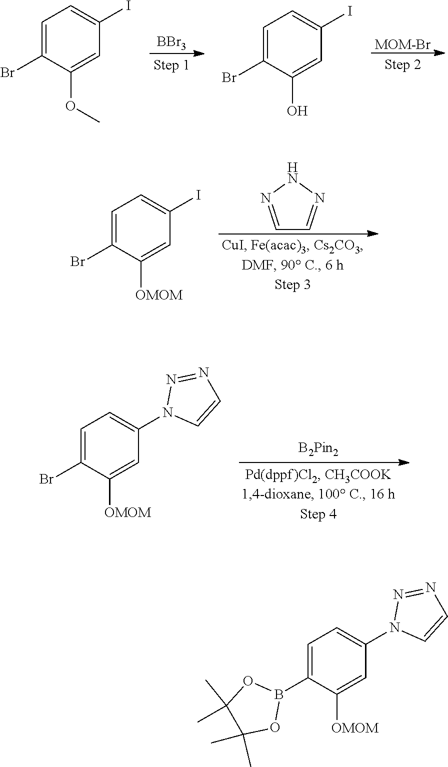

Step 1. To a solution of 1-bromo-4-iodo-2-methoxy-benzene (100 g, 319 mmol) in 100 mL DCM was added BBr₃ (1 M in DCM, 600 mL, 600 mmol). The mixture was stirred at rt for 16 h then poured onto crushed ice and extracted with DCM (200 mL×3). The combined organic phase was concentrated and purified by flash column chromatography (PE/EtOAc=10:1) to give 2-bromo-5-iodo-phenol (90 g, 94.2% yield).

Step 2. To a solution of NaH (60% in mineral oil, 25 g, 625 mmol) in 400 mL THF at 0° C. was added 2-bromo-5-iodo-phenol (92 g, 308 mmol) in 100 mL THF dropwise. After addition, the mixture was stirred for 30 min at 0° C., and then MOMBr (46 g, 368 mmol) was added. The mixture was stirred for another 5-10 min at 0° C. before being quenched with 5% citric acid and concentrated. The residue was mixed with 500 mL DCM, washed with water and brine, dried over Na₂SO₄, and purified by flash column chromatography (PE/EtOAc=20:1) to give 1-bromo-4-iodo-2-(methoxymethoxy)benzene (110 g, 100% yield).

Step 3. To a solution of 1-bromo-4-iodo-2-(methoxymethoxy)benzene (110 g, 321 mmol) in 500 mL DMF were added 1H-1,2,3-triazole (35 g, 507 mmol), Cs₂CO₃ (210 g, 645 mmol), CuI (6.5 g, 34 mmol), and ferric acetylacetonate (34 g, 96 mmol). The mixture was stirred at 90° C. for 6 h before cooling to rt. The mixture was filtered, and the filtrate was concentrated and purified by flash column chromatography (PE/EtOAc=2:1) to give 1-(4-bromo-3-(methoxymethoxy)phenyl)-1H-1,2,3-triazole (25 g, 27.4% yield).

Step 4. To a solution of 1-(4-bromo-3-(methoxymethoxy)phenyl)-1H-1,2,3-triazole (25 g, 88 mmol) in 250 mL 1,4-dioxane were added bis(pinacolato)diboron (38 g, 150 mmol), KOAc (17.5 g, 178 mmol) and Pd(dppf)Cl₂ (6.5 g, 8.9 mmol). The reaction was stirred at 100° C. under Ar for 20 h before concentration. The residue was purified by flash column chromatography (PE/EtOAc=1.5:1) to give 1-(3-(methoxymethoxy)-4-(4,4,5,5-tetramethyl-1,3,2-dioxaborolan-2-yl)phenyl)-1H-1,2,3-triazole (20 g, 68.6%).

### Preparation of 1-[3-Fluoro-5-methoxy-4-(4,4,5,5-tetramethyl-1,3,2-dioxaborolan-2-yl)phenyl]triazole

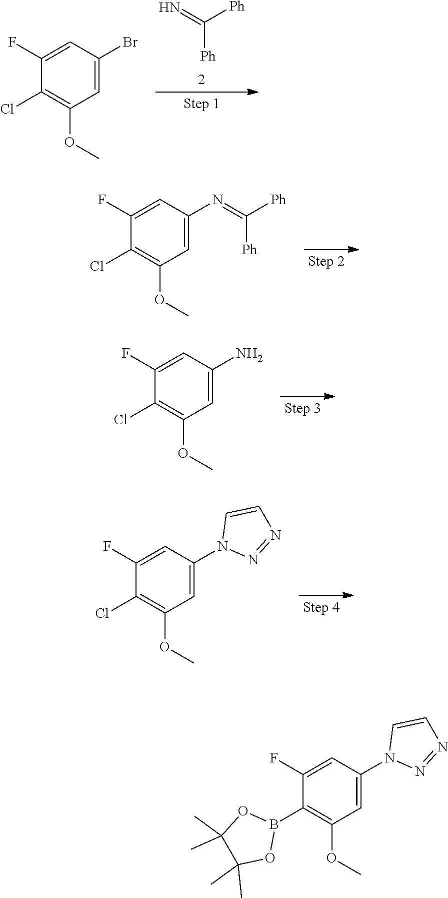

Step 1. To a solution of 5-bromo-2-chloro-1-fluoro-3-methoxy-benzene (1.0 g, 4.2 mmol), diphenylmethanimine (1.05 g, 5.79 mmol), tris(dibenzylideneacetone)dipalladium (0.39 g, 0.42 mmol), RuPhos (0.4 g, 0.84 mmol) in toluene (10 mL) was added sodium tert-butoxide (0.8 g, 8.2 mmol) at 25° C. under nitrogen protection. The mixture was stirred for 16 h at 100° C. After the reaction was complete, the mixture was extracted with EtOAc and washed with brine. The organic layer was dried over Na₂SO4, concentrated and purified by silica gel chromatography (PE:EtOAc=5:1) to give N-(4-chloro-3-fluoro-5-methoxy-phenyl)-1,1-diphenyl-methanimine (800 mg, 2.35 mmol, 56.3% yield) as a colorless oil. MS m/z 340.3 [M+H]⁺.

Step 2. To a solution of N-(4-chloro-3-fluoro-5-methoxy-phenyl)-1,1-diphenyl-methanimine (700 mg, 2.06 mmol) in tetrahydrofuran (5 mL) was added 2 mol/L hydrochloric acid (1 mL), and the mixture was stirred at 25° C. for 1 h. After the reaction was complete, Na₂CO₃ was added to adjust the pH to 9, and the mixture was extracted with EtOAc, dried over Na₂SO₄, concentrated and purified by silica gel chromatography (PE:EtOAc=5:1) to give 4-chloro-3-fluoro-5-methoxy-aniline (300 mg, 82.9% yield) as a light yellow oil. MS m/z 176.2 [M+H]⁺.

Step 3. A solution of 4-methylbenzenesulfonhydrazide (284 mg, 1.49 mmol) and 2,2-dimethoxyacetaldehyde (259 mg, 1.49 mmol, 60 mass % in H₂O) in methanol (5 mL) was stirred at 25° C. for 1 h. Then 4-chloro-3-fluoro-5-methoxy-aniline (250 mg, 1.42 mmol) and acetic acid (89 mg, 1.42 mmol) were added in succession. The mixture was stirred at 75° C. overnight. After the reaction was complete, the solvent was removed by evaporation. The residue was purified by column chromatography (0-50% EtOAc in PE) to afford product 1-(4-chloro-3-fluoro-5-methoxy-phenyl)triazole (200 mg, 61.7% yield). MS m/z 228.1 [M+H]⁺.

Step 4. A solution of 1-(4-chloro-3-fluoro-5-methoxy-phenyl)triazole (120 mg, 0.52 mmol), potassium acetate (103 mg, 1.04 mmol), 4,4,5,5-tetramethyl-2-(4,4,5,5-tetramethyl-1,3,2-dioxaborolan-2-yl)-1,3,2-dioxaborolane (200 mg, 0.78 mmol) and tris(dibenzylideneacetone)dipalladium (48 mg, 0.052 mmol), tricyclohexylphosphine (29 mg, 0.104 mmol) in 1,4-dioxane (3 mL) was stirred at 70° C. for 16 hours under N₂. After the reaction was complete, the solvent was removed under vacuum. The crude residue was purified over silica gel using 30%-35% EtOAc/PE to give 1-[3-fluoro-5-methoxy-4-(4,4,5,5-tetramethyl-1,3,2-dioxaborolan-2-yl)phenyl]triazole (80 mg, 47% yield) as a white solid. MS m/z 320.3 [M+H]⁺.

### Preparation of 1-[2-Fluoro-5-(methoxymethoxy)-4-(4,4,5,5-tetramethyl-1,3,2-dioxaborolan-2-yl)phenyl]triazole

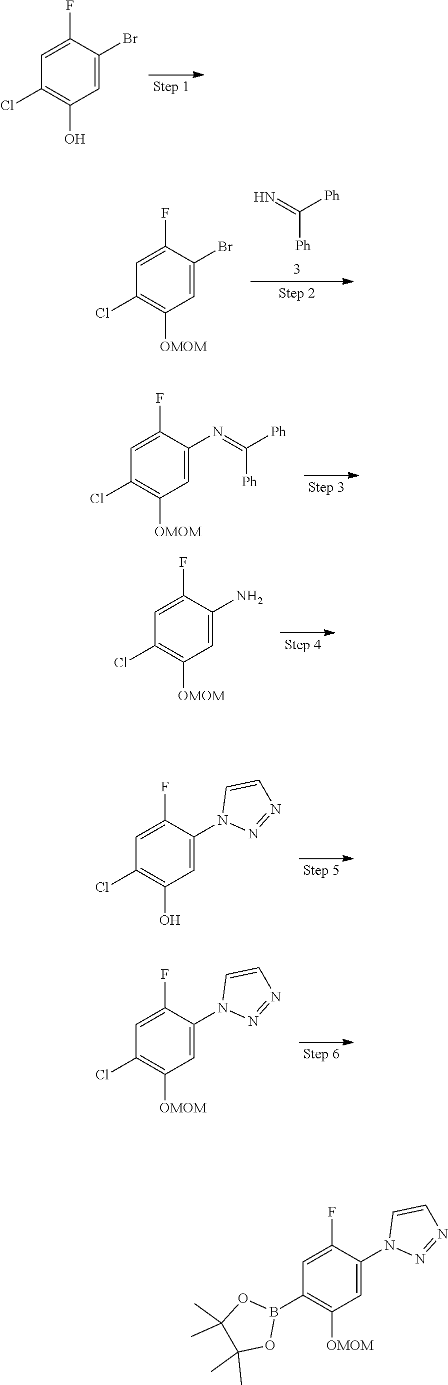

Step 1. To a solution of 5-bromo-2-chloro-4-fluoro-phenol (1 g, 4.4 mmol) in tetrahydrofuran (10 mL) was added sodium hydride (60 mass %) in mineral oil (230 mg, 5.7 mmol) under 0° C. This mixture was stirred at 25° C. for 0.5 h, and then bromomethyl methyl ether (1.1 g, 8.8 mmol) was added. The mixture was stirred at 25° C. for 1 h. After the reaction was complete, the mixture was quenched with water and extracted with EtOAc. The combined organic layers were evaporated to dryness in vacuum and purified by flash column chromatography (PE/EtOAc=20:1) to give 1-bromo-4-chloro-2-fluoro-5-(methoxymethoxy)benzene (800 mg, 66.9% yield) as colorless oil.

Step 2. To a solution of 1-bromo-4-chloro-2-fluoro-5-(methoxymethoxy)benzene (1 g, 3.7 mmol), diphenylmethanimine (1.05 g. 5.79 mmol), tris(dibenzylideneacetone)dipalladium (0.39 g, 0.37 mmol), RuPhos (0.4 g, 0.76 mmol) in toluene (10 mL) was added sodium tert-butoxide (0.8 g. 8.2 mmol) at 25° C. under nitrogen atmosphere. The mixture was stirred for 16 h at 100° C. The mixture was extracted with EtOAc and washed with brine. The organic layer was dried over Na₂SO₄, concentrated and purified by silica gel chromatography to give N-[4-chloro-2-fluoro-5-(methoxymethoxy)phenyl]-1,1-diphenyl-methanimine (0.6 g. 2 mmol, 40% yield) as a colorless oil. MS m/z 370.2 [M+H]⁺.

Step 3. To a solution of N-[4-chloro-2-fluoro-5-(methoxymethoxy)phenyl]-1,1-diphenyl-methanimine (600 mg, 1.6 mmol.) in tetrahydrofuran (5 mL) was added hydrochloric acid (2 mol/L, 2 mL). The mixture was stirred at 25° C. for 1 h. After the reaction was complete, Na₂CO₃ was added to adjust the pH to 9, and the mixture was extracted with EtOAc, dried over Na₂SO₄, concentrated and purified by flash chromatography (PE:EtOAc=4:1) to give 4-chloro-2-fluoro-5-(methoxymethoxy)aniline (0.3 g, 90% yield) as a light yellow oil. MS m/z 206.2 [M+H]⁺.

Step 4. A solution of 4-methylbenzenesulfonhydrazide (194 mg, 0.5 mmol) and 2,2-dimethoxyacetaldehyde in H₂O (177 mg, 0.5 mmol) in methanol (5 mL) was stirred at rt for 1 h. 4-Chloro-2-fluoro-5-(methoxymethoxy)aniline (100 mg. 0.48 mmol) and acetic acid (61 mg, 0.5 mmol) were added in succession. The mixture was stirred at 75° C. overnight. After the reaction was complete, the solvent was removed by evaporation. The residue was purified by column chromatography (0-60% EtOAc in PE) to afford product 2-chloro-4-fluoro-5-(triazol-1-yl)phenol (80 mg. 77% yield). MS m/z 214.1 [M+H]⁺.

Step 5. To a solution of 2-chloro-4-fluoro-5-(triazol-1-yl)phenol (80 mg, 0.37 mmol) in tetrahydrofuran (2 mL) was added sodium hydride (60 mass %) in mineral oil (36 mg, 0.45 mmol) at 0° C. The mixture was stirred at rt for 0.5 h. Bromomethyl methyl ether (112 mg. 0.45 mmol) was added, and the reaction was stirred at rt for 2 h. After the reaction was complete, the mixture was quenched with water and extracted with EtOAc. The combined organic layers were evaporated to dryness in vacuum. The crude residue was purified over silica gel using 15% PE/EtOAc to give 1-[4-chloro-2-fluoro-5-(methoxymethoxy)phenyl]triazole (90 mg, 93% yield) as a light yellow oil. MS m/z 258.1 [M+H]⁺.

Step 6. A solution of 1-[4-chloro-2-fluoro-5-(methoxymethoxy)phenyl]triazole (80 mg, 0.31 mmol), potassium acetate (60 mg, 0.62 mmol), 4,4,5,5-tetramethyl-2-(4,4,5,5-tetramethyl-1,3,2-dioxaborolan-2-yl)-1,3,2-dioxaborolane (118 mg, 0.45 mmol) and [1,1′-bis(diphenylphosphino)ferrocene]dichloropalladium (ii) (24 mg, 0.03 mmol) in 1,4-dioxane (3 mL) was stirred at 80° C. for 16 hours under N₂. The reaction solution was evaporated in vacuo. The crude residue was purified over silica gel using 30%-35% EtOAc/PE to give 1-[2-fluoro-5-(methoxymethoxy)-4-(4,4,5,5-tetramethyl-1,3,2-dioxaborolan-2-yl)phenyl]triazole (90 mg, 83% yield) as a white solid. MS m/z 350.2 [M+H]⁺.

### Preparation of 1-(3-(methoxymethoxy)-4-(4,4,5,5-tetramethyl-1,3,2-dioxaborolan-2-yl)phenyl)-4-methyl-1H-1,2,3-triazole

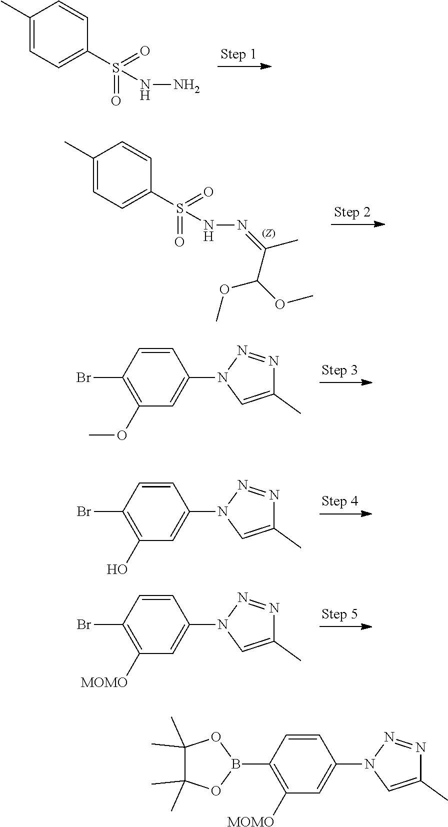

Step 1. To a solution of 4-methylbenzenesulfonohydrazide (500 mg, 2.7 mmol) in methanol (5.0 mL) was added 1,1-dimethoxypropan-2-one (350 mg, 2.9 mmol). The reaction was stirred for 10 min at rt. The material used directly in the next step.

Step 2. To the mixture from Step 1 was added 4-bromo-3-methoxy-aniline (586 mg, 2.9 mmol) and N,N-diisopropylethylamine (0.56 mL, 3.2 mmol). The reaction mixture was heated to 140° C. for 10 min, then cooled to rt and stirred for 16 h at rt. The mixture was partitioned between brine and DCM, and extracted with DCM three times. The combined organic layers were dried over sodium sulfate and concentrated under reduced pressure. The residue was purified by flash column chromatography eluting with a gradient hexane/EtOAc (0-100% EtOAc) to afford 1-(4-bromo-3-methoxy-phenyl)-4-methyl-triazole (610 mg, 86% yield), MS m/z 270.0 [M+H]⁺; ¹H NMR (chloroform-d) δ: 7.74-7.87 (m, 1H), 7.66-7.71 (m, 1H), 7.42-7.50 (m, 1H), 7.04-7.15 (m, 1H), 4.02 (s, 3H), 2.50 (s, 3H).

Step 3. A solution of 1-(4-bromo-3-methoxy-phenyl)-4-methyl-triazole (610 mg, 2.27 mmol), in dichloromethane (2.0 mL) was cooled to −78° C. Boron tribromide (4.5 mL, 4.5 mmol, 1.0 M in DCM) was added dropwise. The reaction was slowly warmed to rt and stirred at rt for 16 h. The reaction was quenched by dropwise addition of aq. sat. NaHCO₃, and extracted with EtOAc 3 times. The combined organic layers were dried over sodium sulfate and concentrated under reduced pressure. The residue was purified by flash column chromatography eluting with a gradient DCM/MeOH (0-30% MeOH) to afford 2-bromo-5-(4-methyltriazol-1-yl)phenol (305 mg, 52.7% yield). MS m/z 256.0 [M+H]⁺

Step 4. To solution of 2-bromo-5-(4-methyltriazol-1-yl)phenol (305 mg, 1.20 mmol) in DMF (6.0 mL) was added N,N-diisopropylethylamine (0.3 mL, 1.80 mmol). The reaction mixture was cooled to 0° C. and chloro(methoxy)methane (0.12 mL, 1.44 mmol) was added. The reaction was stirred at 0° C. for 2 hours, then partitioned between brine and EtOAc. The combined organic layers were dried over sodium sulfate and concentrated under reduced pressure. The residue was purified by flash column chromatography eluting with a gradient hexane/EtOAc (0-100% EtOAc) to afford 1-[4-bromo-3-(methoxymethoxy)phenyl]-4-methyl-triazole (325 mg, 90.8% yield). MS m/z 299.8 [M+H]⁺; ¹H NMR (chloroform-d) δ: 7.74 (s, 1H), 7.67-7.71 (m, 1H), 7.57-7.63 (m, 1H), 7.23-7.28 (m, 1H), 5.35 (s, 2H), 3.56 (s, 3H), 2.47 (s, 3H).

Step 5. To a dry screw cap vial were added: 1-[4-bromo-3-(methoxymethoxy)phenyl]-4-methyl-triazole (325 mg, 1.1 mmol), 4,4,5,5-tetramethyl-2-(4,4,5,5-tetramethyl-1,3,2-dioxaborolan-2-yl)-1,3,2-dioxaborolane (414 mg, 1.63 mmol), [1,1′-bis(diphenylphosphino)ferrocene]dichloropalladium (II) (73 mg, 0.10 mmol) and potassium acetate (301 mg. 2.18 mmol). The mixture was degassed with argon for 10 min, then dioxane (2 mL) and water (0.5 mL) were added. The reaction was heated at 90° C. for 5 h. The reaction was cooled and partitioned between water and ethyl acetate. The combined organic lauers were dried over sodium sulfate and concentrated under reduced pressure. The residue was purified by flash column chromatography eluting with a gradient hexane/EtOAc (0-100% EtOAc) to afford 1-[3-(methoxymethoxy)-4-(4,4,5,5-tetramethyl-1,3,2-dioxaborolan-2-yl)phenyl]-4-methyl-triazole (275 mg, 73.0% yield). MS m/z 346.1 [M+H]⁺; ¹H NMR (chloroform-d) δ: 7.79-7.84 (m, 1H), 7.70-7.78 (m, 1H), 7.42-7.51 (m, 1H), 7.30-7.37 (m, 1H), 5.27 (s, 2H), 3.53 (s, 3H), 2.43 (s, 3H), 1.36 (s, 12H).

### Preparation of 4-chloro-1-(3-methoxy-4-(4,4,5,5-tetramethyl-1,3,2-dioxaborolan-2-yl)phenyl)-1H-1,2,3-triazole

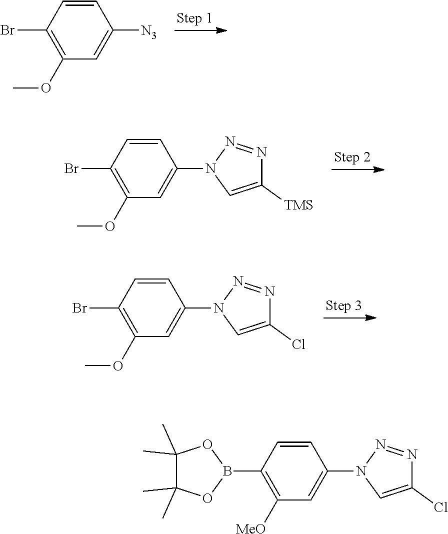 [SMILES: COc1cc(N=[N+]=[N-])ccc1Br ; COc1cc(-n2cc([Si](C)(C)C)nn2)ccc1Br ; COc1cc(-n2cc(Cl)nn2)ccc1Br ; COc1cc(-n2cc(Cl)nn2)ccc1B1OC(C)(C)C(C)(C)O1]

Step 1. To a dry screw cap vial were added: 4-azido-1-bromo-2-methoxy-benzene (1.0 g, 4.4 mmol), cuprous iodide (82 mg, 0.43 mmol), N,N-diisopropylethylamine (0.38 mL, 2.2 mmol) and ACN (3.0 mL). The vial was purged with Ar and ethynyl(trimethyl)silane (1.3 g, 13.1 mmol) was added. The resulting mixture was stirred for at rt for 3 days. Upon completion, the reaction was partitioned between EtOAc and brine. The combined organic layers were dried over sodium sulfate and concentrated under reduced pressure. The residue was purified by flash column chromatography eluting with a gradient hexane/EtOAc (0-100% EtOAc) to afford [1-(4-bromo-3-methoxy-phenyl)triazol-4-yl]-trimethyl-silane (1.3 g, 91% yield) MS m/z 328 [M+H]⁺

Step 2. To a solution of [1-(4-bromo-3-methoxy-phenyl)triazol-4-yl]-trimethyl-silane (800 mg, 2.45 mmol) in ACN (6.0 mL) were added CsF (547 mg, 3.6 mmol) and N-chlorosuccinimide (100 mg, 7.35 mmol). The mixture was heated to 90° C. and stirred for 16 h. The reaction was partitioned between EtOAc and brine. The Combined organic layers were dried over sodium sulfate and concentrated under reduced pressure. The residue was purified by flash column chromatography eluting with a gradient hexane/EtOAc (0-100% EtOAc) to afford 1-(4-bromo-3-methoxy-phenyl)-4-chloro-triazole (385 mg, 54% yield). MS m/z 290.1, 292.1 [M+H]⁺; ¹H NMR (chloroform-d) δ: 7.96 (s, 1H), 7.71 (d, J=8.5 Hz, 1H), 7.43 (d, J=2.4 Hz, 1H), 7.09 (dd, J=8.5, 2.4 Hz, 1H), 4.02 (s, 3H).

Step 3. To a dry screw cap vial were added: 1-(4-bromo-3-methoxy-phenyl)-4-chloro-triazole (385 mg, 1.3 mmol), bis(pinacolato)diboron (675 mg, 2.7 mmol), potassium acetate (367 mg, 2.7 mmol) and [1,1′-bis(diphenylphosphino)ferrocene] dichloropalladium (II) (95 mg, 0.13 mmol). The mixture was degassed with argon for 10 min, then dioxane (2 mL) and water (0.5 mL) were added. The reaction was heated at 90° C. for 7 h. The reaction was cooled and partitioned between water and ethyl acetate. The combined organic layers were dried over sodium sulfate and concentrated under reduced pressure. The residue was purified by flash column chromatography eluting with a gradient hexane/EtOAc (0-100% EtOAc) to afford (400 mg, 89% yield) as a brownish oil. Product doesn't ionize on LC/MS.

### Preparation of (2-(methoxymethoxy)-4-(5-methyl-1H-1,2,3-triazol-1-yl)phenyl)boronic acid

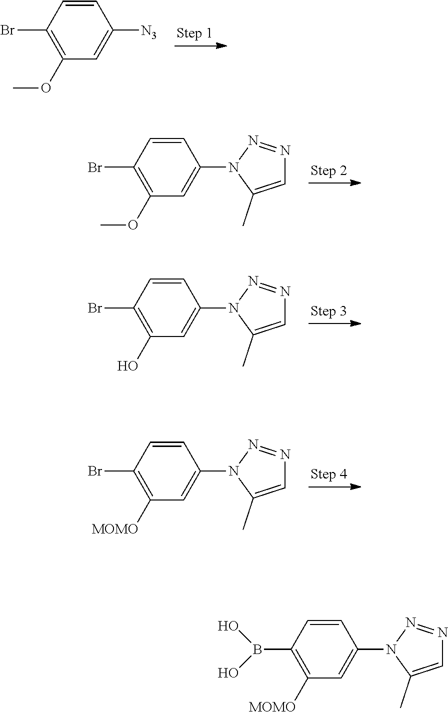

Step 1. To a solution of 4-azido-1-bromo-2-methoxy-benzene (3 g, 13.15 mmol) in ACN (6.0 mL) was added 1,1,3,3-tetramethylguanidine (2.3 g, 19.7 mmol) and 1-dimethoxyphosphorylpropan-2-one (3.3 g, 19.73 mmol). The reaction was heated to 80° C. for 2 h. The reaction was partitioned between EtOAc and brine. The Combined organic layers were dried over sodium sulfate and concentrated under reduced pressure. The residue was purified by flash column chromatography eluting with a gradient DCM/EtOAc (0-100% EtOAc) to afford 1-(4-bromo-3-methoxy-phenyl)-5-methyl-triazole (1.1 g, 31% yield). MS m/z 268.1, 270.1 [M+H]⁺; ¹H NMR (chloroform-d) δ: 7.62 (d, J=8.4 Hz, 1H), 7.51 (s, 1H), 7.01 (d, J=2.0 Hz, 1H), 6.83 (dd, J=8.4, 2.0 Hz, 1H), 3.87 (s, 3H), 2.30 (s, 3H).

Step 2. A solution of 1-(4-bromo-3-methoxy-phenyl)-5-methyl-triazole (1.1 g, 4.1 mmol) in DCM (10 mL) was cooled to −78° C. Boron tribromide (0.77 mL, 8.2 mmol) was added dropwise. The reaction was slowly warmed to rt and stirred at rt for 3, then quenched by dropwise addition of aq. sat. NaHCO₃ and extracted with EtOAc three times. The combined organic layers were dried over sodium sulfate and concentrated under reduced pressure to afford crude 2-bromo-5-(5-methyltriazol-1-yl)phenol that was used for the next step without further purification.

Step 3. To a solution of 2-bromo-5-(5-methyltriazol-1-yl)phenol (1.0 g, 3.9 mmol) in DMF (10 mL) was added N,N-diisopropylethylamine (1.0 mL, 5.9 mmol). The mixture was cooled to −78° C. Chloro(methoxy)methane (378 mg, 4.7 mmol) was added dropwise. The reaction was warmed to 0° C. and stirred for 2 min at this temperature. The reaction was partitioned between EtOAc and brine. The combined organic layers were dried over sodium sulfate and concentrated under reduced pressure. The residue was purified by flash column chromatography eluting with a gradient hexane/EtOAc (0-100% EtOAc) to afford 1-[4-bromo-3-(methoxymethoxy)phenyl]-5-methyl-triazole (550 mg, 47% yield). MS m/z 298.1, 300.1 [M+H]⁺; ¹H NMR (chloroform-d) δ: 7.65-7.79 (m, 1H), 7.48-7.63 (m, 1H), 7.27-7.38 (m, 1H), 6.93-7.10 (m, 1H), 5.30 (s, 2H), 3.52 (s, 3H), 2.37 (s, 3H).

Step 4. To a dry screw cap vial were added: 1-[4-bromo-3-(methoxymethoxy)phenyl]-5-methyl-triazole (550 mg, 1.84 mmol), 4,4,5,5-tetramethyl-2-(4,4,5,5-tetramethyl-1,3,2-dioxaborolan-2-yl)-1,3,2-dioxaborolane (700 mg, 2.76 mmol), XPhos Pd G3 (68 mg, 0.14 mmol) and potassium acetate (636 mg, 4.6 mmol). The mixture was degassed with argon for 10 min, then dioxane (2 mL) and water (0.5 mL) were added. The reaction was heated at 90° C. for 5 h. The reaction was cooled and partitioned between water and ethyl acetate. The combined organic layers were dried over sodium sulfate and concentrated under reduced pressure. The residue was purified by flash column chromatography eluting with a gradient hexane/EtOAc (0-100% EtOAc) to afford [2-(methoxymethoxy)-4-(5-methyltriazol-1-yl)phenyl]boronic acid (240 mg, 49% yield). MS m/z 264 [M+H]⁺; ¹H NMR (methanol-d₄) δ: 7.57-7.75 (m, 2H), 7.28-7.36 (m, 1H), 7.13-7.24 (m, 1H), 5.32 (s, 2H), 3.51 (s, 3H), 2.40 (s, 3H), 1.93 (s, 3H).

### Example 1

### Preparation of Compound 8

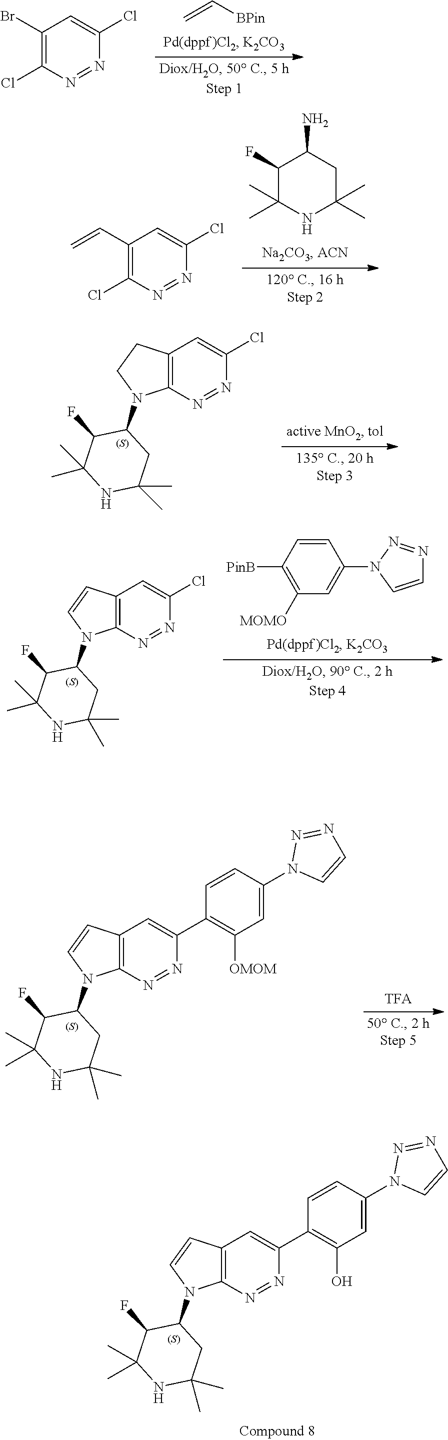

Step 1. To a solution of 4-bromo-3,6-dichloro-pyridazine (26.0 g, 114.1 mmol) in 260 mL 1,4-dioxane and 65 mL water was added 4,4,5,5-tetramethyl-2-vinyl-1,3,2-dioxaborolane (18.5 g, 120.1 mmol), K₂CO₃ (31.5 g, 228.3 mmol) and [1,1′-bis(diphenylphosphino)ferrocene]dichloropalladium (II) (4.25 g, 5.7 mmol). The mixture was stirred at 50° C. under N₂ for 5 h before concentration. The residue was purified by flash column chromatography (PE/EtOAc=4:1) to give 3,6-dichloro-4-vinyl-pyridazine (12.5 g, 58.3%) as white solid. MS m/z 175.1, 176.1 [M+H]⁺.

Step 2. A mixture of 3,6-dichloro-4-vinyl-pyridazine (5.0 g, 28.6 mmol), Na₂CO₃ (3.1 g, 29.2 mmol), (3S,4S)-3-fluoro-2,2,6,6-tetramethyl-piperidin-4-amine (5.5 g, 31.6 mmol) in 25 mL acetonitrile was heated at 120° C. under N₂ for 16 h. After being cooled to room temperature, the mixture was concentrated. The residue was purified by flash column chromatography (DCM/MeOH=20:1) to give 3-chloro-7-[(3S,4S)-3-fluoro-2,2,6,6-tetramethyl-4-piperidyl]-5,6-dihydropyrrolo[2,3-c]pyridazine (6.0 g, 67.1%) as brown foam. MS m/z 313.4, 315.4 [M+H]⁺.

Step 3. To a sealed tube were added 3-chloro-7-[(3S,4S)-3-fluoro-2,2,6,6-tetramethyl-4-piperidyl]-5,6-dihydropyrrolo[2,3-c]pyridazine (6.0 g, 19.2 mmol), 300 mL anhydrous toluene, active MnO₂ (84 g. 966.2 mmol) and 2.0 g 4 Å molecular sieves (freshly dried at high temperature). This mixture was stirred at 135° C. for 16 h before being cooled to room temperature. The solid materials were removed by filtration, and the filtrate was concentrated. The residue was purified by flash column chromatography (DCM/MeOH=20:1) to give 3-chloro-7-[(3S,4S)-3-fluoro-2,2,6,6-tetramethyl-4-piperidyl]pyrrolo[2,3-c]pyridazine (3.9 g. 65.4%) as brown solid. MS m/z 311.4, 313.4 [M+H]⁺.

Step 4. To a dry screw cap vial were added: 3-chloro-7-((3S,4S)-3-fluoro-2,2,6,6-tetramethylpiperidin-4-yl)-7H-pyrrolo[2,3-c]pyridazine (50 mg, 0.16 mmol), 1-(3-(methoxymethoxy)-4-(4,4,5,5-tetramethyl-1,3,2-dioxaborolan-2-yl)phenyl)-1H-1,2,3-triazole (85 mg. 0.25 mmol), XPhos Pd G4 (0.14 mg, 0.016 mmol) and K₂CO₃ (66 mg. 0.48 mmol). The mixture was degassed with argon for 10 min, then dioxane (2 mL) and water (0.5 mL) were added. The reaction was heated at 90° C. for 5 h. The reaction was cooled to rt, partitioned between EtOAc and water. The organic layers were dried over Na₂SO₄, concentrated, and purified by silica-gel column chromatography eluting with a gradient (0-10%) CH₂Cl₂/MeOH to afford 7-((3S,4S)-3-fluoro-2,2,6,6-tetramethylpiperidin-4-yl)-3-(2-(methoxymethoxy)-4-(1H-1,2,3-triazol-1-yl)phenyl)-7H-pyrrolo[2,3-c]pyridazine (40 mg, 52% yield). MS m/z 480.5 [M+H]⁺; ¹H NMR (500 MHz, methanol-d₄) δ: 8.66 (s, 1H), 8.29 (s, 1H), 8.02 (br s, 1H), 7.96 (s, 1H), 7.83-7.91 (m, 2H), 7.66 (d, J=8.24 Hz, 1H), 6.71 (m, 1H), 5.89 (m, 1H), 5.37 (s, 2H), 4.58 (m, 1H), 3.44 (s, 3H), 2.36 (d. J=13.12 Hz, 1H), 1.90 (dd, J=12.44, 3.74 Hz, 1H), 1.51 (m, 3H), 1.47 (s, 3H), 1.35 (s, 3H), 1.28 (s, 3H); 1H not observed (NH).

Step 5. To a solution of 2-(7-((3S,4S)-3-fluoro-2,2,6,6-tetramethylpiperidin-4-yl)-7H-pyrrolo[2,3-c]pyridazin-3-yl)-5-(1H-1,2,3-triazol-1-yl)phenol (40 mg, 0.083 mmol) in CH₂Cl₂ (1 mL) and 2 drops of MeOH was added HCl (4 mol/L) in 1,4-dioxane (0.1 mL, 0.4 mmol). The reaction was stirred for 2 h. The solvents were removed under reduced pressure, and the residue was purified by silica-gel column chromatography eluting with a gradient (0-30%) CH₂C₁₂/MeOH (containing 2.5% NH₄OH) to afford 2-(7-((3S,4S)-3-fluoro-2,2,6,6-tetramethylpiperidin-4-yl)-7H-pyrrolo[2,3-c]pyridazin-3-yl)-5-(1H-1,2,3-triazol-1-yl)phenol (25 mg, 69% yield) as a tan solid. MS m/z 436.4 [M+H]⁺; ¹H NMR (500 MHz, methanol-d₄) δ: 8.89 (s, 1H), 8.67 (m, 2H), 7.97 (m, 2H), 7.72 (s, 1H), 7.65 (br d, J=8.24 Hz, 1H), 7.18 (d, J=2.90 Hz, 1H), 5.90 (m, 1 H), 5.12 (d, J=50.96 Hz, 1H), 2.88 (br t, J=13.50 Hz, 1H), 2.41 (br d, J=11.44 Hz, 1H), 1.82 (s, 3H), 1.78 (s, 3H), 1.69 (s, 3H), 1.65 (s, 3H); 2Hs not observed.

Using the procedure described for Example 1, above, additional compounds described herein may be prepared by substituting the appropriate starting material, suitable reagents and reaction conditions, obtaining compounds such as those selected from:

| Cpd | Data |
|---|---|
| 1 | MS m/z 418.4 [M + H]⁺; ¹H NMR (methanol-d₄) δ: 8.63 (s, 1H), 8.62 (s, |
| | 1H), 8.19 (d, J = 8.4 Hz, 1H), 8.04 (d, J = 3.5 Hz, 1H), 7.94 (s, 1H), |
| | 7.50-7.54 (m, 2H), 6.77 (d, J = 3.4 Hz, 1H), 5.51 (tt, J = 12.6, 3.5 Hz, |
| | 1H), 2.12 (dd, J = 12.5, 3.2 Hz, 2H), 1.98-2.00 (m, 1H), 1.92-2.04 (m, |
| | 1H), 1.50 (s, 6H), 1.32 (s, 6H); 2Hs not observed (NH and OH) |
| 2 | MS m/z 436.4 [M + H]⁺; ¹H NMR (methanol-d₄) δ: 8.69 (s, 1H), 8.63 (s, |
| | 1H), 8.17 (d, J = 7.8 Hz, 1H), 8.08 (br s, 1H), 7.94 (s, 1H), 7.55 (s, 2H), |
| | 6.79 (d, J = 3.2 Hz, 1H), 5.80-5.99 (m, 1H), 5.04 (d, J = 50.96 Hz, 1H), |
| | 2.45-2.69 (m, 1H), 1.97-2.27 (m, 1H), 1.67 (br s, 3H), 1.63 (br s, 3H), |
| | 1.51 (br s, 3H), 1.46 (br s, 3H); 2Hs not observed (NH and OH) |
| 3 | MS m/z 436.5 [M + H]⁺; ¹H NMR (methanol-d₄) δ: 8.67 (s, 1H), 8.62 (s, |
| | 1H), 8.21 (d, J = 8.2 Hz, 1H), 8.07 (br s, 1H), 7.94 (s, 1H), 7.48-7.56 |
| | (m, 2H), 6.79 (d, J = 3.4 Hz, 1H), 5.76-5.90 (m, 1H), 4.55 (d, J = 50.5 |
| | Hz, 1H), 2.37 (br t, J = 12.9 Hz, 1H), 1.92 (br dd, J = 12.4, 3.6 Hz, 1H), |
| | 1.53 (s, 3H), 1.48 (s, 3H), 1.36 (s, 3H), 1.29 (br s, 3H); 2Hs not |
| | observed (NH and OH) |
| 4 | MS m/z 388.3 [M + H]⁺; ¹H NMR (DMSO-d₆) δ ppm: 9.78 (s, 1H), 9.37 |
| | (s, 1H) 8.94 (s, 1H) 8.83 (m, 1H), 8.31 (s, 1H), 8.31 (m, 1H), 8.18 (m, |
| | 1H), 8.02 (s, 1H) 7.65 (s, 1H), 7.52 (m, 1H), 6.89 (m, 1H), 5.21 (tt, J = |
| | 11.90, 3.97 Hz, 1H), 3.53 (br d, J = 12.51 Hz, 1H), 3.27 (m, 1H), 2.93 |
| | (m, 1H), 2.57 (m, 1H), 2.38 (m, 1H), 1.78 (br d, J = 13.12 Hz, 1H), |
| | 1.19 (m, 2H), 0.89 (m, 2H) |
| 5 | MS m/z 390.4 [M + H]⁺; ¹H NMR (methanol-d₄) δ: 8.66 (s, 1H), 8.61 (s, |
| | 1H), 8.19 (d, J = 8.5 Hz, 1H), 7.98 (d, J = 3.6 Hz, 1H), 7.93 (s, 1H), |
| | 7.54-7.50 (m, 2H), 6.79 (d, J = 3.5 Hz, 1H), 5.34 (tt, J = 12.3, 4.1 Hz, |
| | 1H), 3.57-3.45 (m, 2H), 2.57-2.51 (m, 2H), 2.41 (br. d, J = 13.6 Hz, |
| | 1H), 2.31 (br. dd, J = 13.8, 3.7 Hz, 1H), 1.62 (s, 3H), 1.52 (s, 3H) |
| 6 | MS m/z 424.3 [M + H]⁺; ¹H NMR (methanol-d₄) δ: 8.58 (d, J = 1.2 Hz, |
| | 1H), 8.07-7.96 (m, 2H), 7.91 (s, 1H), 7.47-7.45 (m, 2H), 5.08 (d, J = |
| | 49.4 Hz, 1H), 4.54-4.40 (m, 1H), 3.61-3.37 (m, 4H), 2.85 (dd, J = |
| | 18.8, 4.2 Hz, 1H), 2.33 (t, J = 13.3 Hz, 1H), 1.94-1.88 (m, 1H), 1.41 |
| | (s, 9H) |
| 7 | MS m/z 402.3 [M + H]⁺; ¹H NMR (DMSO-d₆) δ: 11.10 (br s, 1H), 8.94 |
| | (s, 1H), 8.82 (s, 1H), 8.62 (br s, 1H), 8.18 (br d, J = 8.54 Hz, 1H), 8.03 |
| | (s, 1H) 7.66 (s, 1H), 7.58 (m, 1H), 6.94 (br d, J = 2.75 Hz, 1H), 5.28 (m, |
| | 1H), 3.62 (m, 1H), 3.45 (m, 1H), 3.07 (s, 3H), 3.03 (m, 1H), 2.67 (m, |
| | 1H), 2.27 (m, 1H), 1.57 (m, 2H), 1.21 (m, 1H), 1.02 (m, 1H), 0.87 (m, |
| | 1H) |
| 9 | MS m/z 402.3 [M + H]⁺; ¹H NMR (methanol-d₄) δ: 8.61 (s, 1 H), 8.46 (s, |
| | 1 H), 8.16 (br, J = 8.24 Hz, 1 H), 7.91-7.98 (m, 1 H), 7.45-7.59 (m, 2 |
| | H), 6.53 (s, 1 H), 3.80 (t, J = 12.82 Hz, 1 H), 3.65 (d, J = 11.60 Hz, 1 |
| | H), 3.34-3.52 (m, 3 H), 2.68 (s, 3 H), 2.29 (d, J = 13.12 Hz, 2 H), 1.65 |
| | (dd, J = 13.73, 2.14 Hz, 2 H), 1.04-1.14 (m, 1 H), 0.95-1.02 (m, 1 H) |
| 11 | MS m/z 388. [M + H]⁺; ¹H NMR (methanol-d₄) δ: 8.65 (s, 1H), 8.62 (s, |
| | 1H), 8.27 (s, 1H), 8.19 (d, J = 8.5 Hz, 1H), 8.01 (d, J = 3.5 Hz, 1H), |
| | 7.94 (s, 1H), 7.56-7.49 (m, 2H), 6.79 (d, J = 3.5 Hz, 1H), 5.29 (tt, J = |
| | 12.2, 4.2 Hz, 1H), 3.66 (dt, J = 12.9, 3.5 Hz, 1H), 3.46 (td, J = 12.9, 3.1 |
| | Hz, 1H), 3.06 (t, J = 12.9 Hz, 1H), 2.64 (dd, J = 13.0, 4.3 Hz, 1H), 2.54- |
| | 2.47 (m, 1H), 1.88 (dt, J = 13.6, 3.2 Hz, 1H), 1.20-1.09 (m, 3H), |
| | 1.05-0.95 (m, 1H) |
| 12 | MS m/z 408.5 [M + H]⁺; ¹H NMR (DMSO-d₆) δ: 14.27 (s, 1H), 8.93 (s, |
| | 1H), 8.82 (s, 1H), 8.29 (d, J = 8.6 Hz, 1H), 8.15 (dd, J = 3.5, 1.9 Hz, |
| | 1H), 8.00 (s, 1H), 7.61-7.53 (m, 2H), 6.77 (d, J = 3.5 Hz, 1H), 5.43 |
| | (dddd, J = 32.2, 13.6, 4.5, 2.1 Hz, 1H), 4.87 (d, J = 51.0 Hz, 1H), 3.29- |
| | 3.11 (m, 2H), 2.25 (t, J = 13.0 Hz, 1H), 1.81 (dd, J = 12.3, 4.4 Hz, 1H), |
| | 1.26 (s, 3H), 1.22 (s, 3H) |
| 13 | MS m/z 388.4 [M + H]⁺; ¹H NMR (methanol-d₄) δ: 8.77 (s, 1H), 8.64 (s, |
| | 1H), 8.44 (d, J = 3.5 Hz, 1H), 8.01-7.95 (m, 2H), 7.65 (d, J = 2.1 Hz, |
| | 1H), 7.59 (dd, J = 8.5, 2.2 Hz, 1H), 7.04 (d, J = 3.4 Hz, 1H), 5.34 (tt, J = |
| | 12.1, 4.1 Hz, 1H), 3.71 (ddd, J = 12.8, 4.3, 2.4 Hz, 1H), 3.51 (td, J = |
| | 13.0, 3.3 Hz, 1H), 3.08 (t, J = 12.9 Hz, 1H), 2.67 (qd, J = 13.2, 4.3 Hz, |
| | 1H), 2.56 (dt, J = 13.8, 3.4 Hz, 1H), 1.93 (ddd, J = 13.7, 4.3, 2.1 Hz, |
| | 1H), 1.27-1.18 (m, 2H), 1.18-1.09 (m, 1H), 1.09-1.00 (m, 1H) |
| 20 | MS m/z 406.3 [M + H]⁺; ¹H NMR (DMSO-d₆) δ: 8.92 (s, 1H), 8.82 (s, |
| | 1H), 8.29 (d, J = 8.7 Hz, 1H), 8.16-8.09 (m, 1H), 8.01 (s, 1H), 7.61- |
| | 7.52 (m, 2H), 6.77 (d, J = 3.7 Hz, 1H), 5.49-5.33 (m, 1H), 4.75 (d, J = |
| | 55 Hz, 1H), 3.66-3.62 (m, 2H), 2.50-2.44 (m, 1H), 1.95-1.74 (m, 5H); |
| | 2Hs not observed (NH and OH) |
| 21 | MS m/z 434.3 [M + H]⁺; ¹H NMR (DMSO-d₆) δ: 8.93 (s, 1H), 8.81 (s, |
| | 1H), 8.29 (d, J = 8.7 Hz, 1H), 8.15-8.11 (m, 1H), 8.01 (s, 1H), 7.61- |
| | 7.54 (m, 2H), 6.76 (d, J = 3.5 Hz, 1H), 5.54-5.40 (m, 1H), 4.58 (d, J = |
| | 50.0 Hz, 1H), 2.28-2.23 (m, 1H), 1.91-1.89 (m, 3H), 1.67-1.64 (m, |
| | 2H), 1.28 (s, 3H), 1.21 (s, 3H); 2Hs not observed (NH and OH) |
| 22 | MS m/z 408.3 [M + H]⁺; ¹H NMR (chloroform-d) δ: 8.34 (s, 1H), 8.05 |
| | (d, J = 1.3 Hz, 1H), 7.98 (d, J = 8.8 Hz, 1H), 7.87 (d, J = 1.0 Hz, 1H), |
| | 7.83 (dd, J = 3.5, 2.3 Hz, 1H), 7.51 (dd, J = 8.5, 2.5 Hz, 1H), 7.42 (d, J = |
| | 2.3 Hz, 1H), 6.66 (d, J = 3.5 Hz, 1H), 5.40-5.64 (m, 1H), 4.73-4.97 |
| | (m, 1H), 3.19-3.43 (m, 2H), 2.22 (t, J = 13.1 Hz, 1H), 1.89 (dd, J = 12.3, |
| | 4.3 Hz, 1H), 1.38 (s, 3H), 1.29-1.35 (m, 3H); 2Hs not observed (NH and |
| | OH) |
| 23 | MS m/z 420.1 [M + H]⁺; ¹H NMR (chloroform-d) δ: 8.34 (s, 1H), 8.05 |
| | (d, J = 1.0 Hz, 1H), 7.98 (d, J = 8.8 Hz, 1H), 7.85-7.90 (m, 2H), 7.51 |
| | (dd, J = 8.5, 2.3 Hz, 1H), 7.42 (d, J = 2.3 Hz, 1H), 6.67 (d, J = 3.5 Hz, |
| | 1H), 5.34-5.49 (m, 1H), 4.80-4.97 (m, 1H), 3.17 (ddd, J = 13.6, 4.1, 1.8 |
| | Hz, 1H), 2.99 (td, J = 13.5, 3.5 Hz, 1H), 2.20-2.35 (m, 3H), 2.10-2.18 |
| | (m, 1H), 2.00-2.08 (m, 1H), 1.88-1.98 (m, 3H); 2Hs not observed (NH |
| | and OH) |
| 24 | MS m/z 414.1 [M + H]⁺; ¹H NMR (methanol-d₄) δ: 8.62 (s, 1H), 8.60 (d, |
| | J = 1.3 Hz, 1H), 8.18 (d, J = 8.3 Hz, 1H), 8.04 (d, J = 3.5 Hz, 1H), 7.92 |
| | (d, J = 1.0 Hz, 1H), 7.48-7.54 (m, 2H), 6.74 (d, J = 3.5 Hz, 1H), 5.35- |
| | 5.49 (m, 1H), 2.63 (t, J = 13.1 Hz, 2H), 1.64 (dd, J = 13.1, 4.0 Hz, 2H), |
| | 0.79-0.86 (m, 2H), 0.71-0.77 (m, 2H), 0.62-0.69 (m, 2H), 0.50-0.58 (m, |
| | 2H); 2Hs not observed (NH and OH) |
| 25 | MS m/z 434.2 [M + H]⁺; ¹H NMR (chloroform-d) δ: 8.34 (s, 1H), 8.05 |
| | (d, J = 1.0 Hz, 1H), 7.97 (d, J = 8.8 Hz, 1H), 7.87 (d, J = 1.0 Hz, 1H), |
| | 7.83 (dd, J = 3.5, 2.3 Hz, 1H), 7.50 (dd, J = 8.5, 2.3 Hz, 1H), 7.41 (d, J = |
| | 2.3 Hz, 1H), 6.66 (d, J = 3.5 Hz, 1H), 5.46 (dddd, J = 32.0, 14.3, 4.3, |
| | 2.0 Hz, 1H), 4.88 (d, J = 50.7 Hz, 1H), 3.32-3.42 (m, 1H), 3.15-3.30 (m, |
| | 1H), 2.41 (t, J = 12.8 Hz, 1H), 1.98-2.07 (m, 1H), 1.81-1.93 (m, 3H), |
| | 1.64-1.76 (m, 5H); 2Hs not observed (NH and OH) |
| 26 | MS m/z 454.4 [M + H]⁺; ¹H NMR (chloroform-d) δ: 8.24 (s, 1H), 8.16 |
| | (dd, J = 2.6, 1.2 Hz, 1H), 7.89 (d, J = 1.2 Hz, 1H), 7.86 (dd, J = 3.6, 2.1 |
| | Hz, 1H), 7.74 (d, J = 7.4 Hz, 1H), 7.72 (d, J = 1.8 Hz, 1H), 6.67 (d, J = |
| | 3.6 Hz, 1H), 5.92-5.75 (m, 1H), 4.54 (d, J = 51.6 Hz, 1H), 2.24-2.13 |
| | (m, 1H), 1.90-1.83 (m, 1H), 1.50 (s, 3H), 1.45 (s, 3H), 1.32 (s, 3H), |
| | 1.25 (s, 3H); 2Hs not observed (NH and OH) |
| 27 | MS m/z 454.4 [M + H]⁺; ¹H NMR (chloroform-d) δ: 8.60 (s, 1H), 8.05 |
| | (d, J = 1.2 Hz, 1H), 7.88 (d, J = 1.2 Hz, 1H), 7.85 (dd, J = 3.6, 2.0 Hz, |
| | 1H), 7.34 (dd, J = 13.2, 2.4 Hz, 1H), 7.24 (dd, J = 2.4, 1.3 Hz, 1H), 6.68 |
| | (d, J = 3.6 Hz, 1H), 5.90-5.73 (m, 1H), 4.54 (d, J = 51.6 Hz, 1H), 2.24- |
| | 2.13 (m, 1H), 1.89-1.84 (m, 1H), 1.50 (d, J = 1.6 Hz, 3H), 1.45 (s, |
| | 3H), 1.32 (s, 3H), 1.25 (d, J = 2.0 Hz, 3H); 2Hs not observed (NH and |
| | OH) |

### Example 2

### Preparation of Compound 14

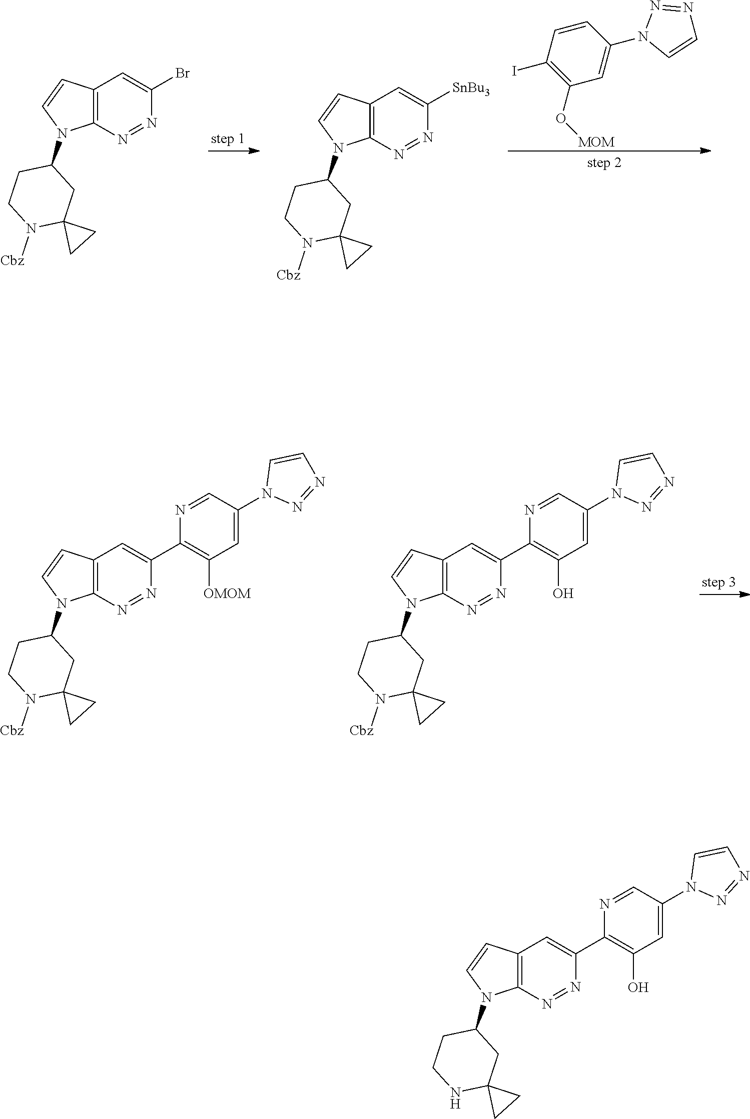

Step 1. A stirred solution of benzyl (7S)-7-(3-bromopyrrolo[2,3-c]pyridazin-7-yl)-4-azaspiro[2.5]octane-4-carboxylate (265 mg, 0.6 mmol) in dry THF (6.0 mL) was cooled to −78° C., then a solution of nBuLi (1.6 mol/L in hexanes, 0.41 mL, 0.66 mmol) was added dropwise. The reaction mixture was stirred for 15 min, then tributyltin chloride (0.20 mL, 0.71 mmol) was added slowly, and the mixture stirred for additional 30 min. The cooling bath was removed, the solution was warmed to rt and stirred for additional 1 hour at rt. The solvent was removed under reduced pressure, toluene (3.0 mL) was added, and the mixture was filtered to remove the precipitate, which was washed with toluene. The filtrate was combined and used in Step 2 without further purification.

Step 2. To the above solution of benzyl (R)-7-(3-(tributylstannyl)-7H-pyrrolo[2,3-c]pyridazin-7-yl)-4-azaspiro[2.5]octane-4-carboxylate was added 2-iodo-3-(methoxymethoxy)-5-(triazol-1-yl)pyridine (100 mg. 0.3 mmol), Pd(dppf)Cl₂ DCM complex (25 mg, 0.03 mmol), Cul (11 mg, 0.058 mmol). The reaction was stirred at 100° C. for 16 h. The reaction was cooled to rt, concentrated, and the residue was purified by reverse phase chromatography eluting with a gradient ACN/H₂O/TFA (0-100% ACN (0.1% TFA)) to afford mixture of benzyl (R)-7-(3-(3-(methoxymethoxy)-5-(1H-1,2,3-triazol-1-yl)pyridin-2-yl)-7H-pyrrolo[2,3-c]pyridazin-7-yl)-4-azaspiro[2.5]octane-4-carboxylate and benzyl (R)-7-(3-(3-hydroxy-5-(1H-1,2,3-triazol-1-yl)pyridin-2-yl)-7H-pyrrolo[2,3-c]pyridazin-7-yl)-4-azaspiro[2.5]octane-4-carboxylate (170 mg), which was used in the next step without further purification

Step 3. The mixture of benzyl (R)-7-(3-(3-(methoxymethoxy)-5-(1H-1,2,3-triazol-1-yl)pyridin-2-yl)-7H-pyrrolo[2,3-c]pyridazin-7-yl)-4-azaspiro[2.5]octane-4-carboxylate from Step 2 and benzyl (R)-7-(3-(3-hydroxy-5-(1H-1,2,3-triazol-1-yl)pyridin-2-yl)-7H-pyrrolo[2,3-c]pyridazin-7-yl)-4-azaspiro[2.5]octane-4-carboxylate was treated with TFA (2.0 mL, 26 mmol) at 60° C. for 2 h. The reaction was cooled to rt, concentrated, and purified by reverse phase chromatography eluting with a gradient ACN/H₂O/TFA (0-100% ACN (0.1% TFA)) to provide 2-[7-[(7S)-4-azaspiro[2.5]octan-7-yl]pyrrolo[2,3-c]pyridazin-3-yl]-5-(triazol-1-yl)pyridin-3-ol;2,2,2-trifluoroacetic acid (14 mg. 9% yield) as a tan foam. MS m/z 389.3 [M+H]⁺; ¹H NMR (500 MHz, methanol-d₄) δ: 9.10 (s, 1H), 8.76-8.87 (m, 1H), 8.71 (s, 1H), 8.00-8.16 (m, 1H), 7.96-8.00 (m, 1H), 7.93 (s, 1H), 6.88 (br d. J=3.1 Hz, 1H), 5.32 (br t, J=11.9 Hz, 1H), 3.69 (br d, J=12.5 Hz, 1H), 3.40-3.58 (m, 1H), 3.32-3.39 (m, 1H), 3.09 (br t, J=12.7 Hz, 1H), 2.60-2.75 (m, 1H), 2.47-2.60 (m, 1H), 1.91 (br d. J=14.0 Hz, 1H), 1.11-1.23 (m, 2H), 0.93-1.11 (m, 1H); 2Hs not observed (NH and OH).

### Example 3

### Preparation of Compound 15

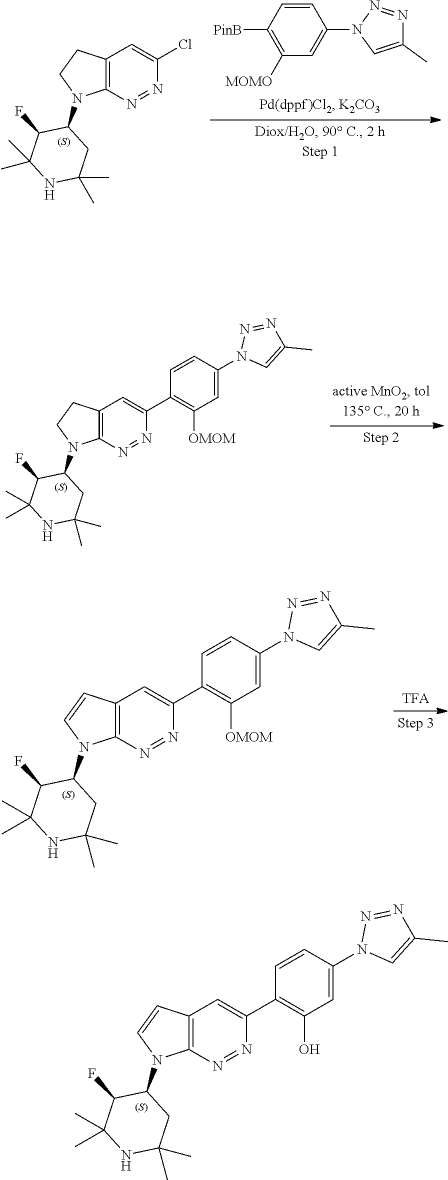

Step 1. To a dry screw cap vial were added: 3-chloro-7-((3S,4S)-3-fluoro-2,2,6,6-tetramethylpiperidin-4-yl)-6,7-dihydro-5H-pyrrolo[2,3-c]pyridazine (50 mg, 0.16 mmol), 1-(3-(methoxymethoxy)-4-(4,4,5,5-tetramethyl-1,3,2-dioxaborolan-2-yl)phenyl)-4-methyl-1H-1,2,3-triazole (56 mg, 0.16 mmol), [1,1′-bis(diphenylphosphino)ferrocene]dichloropalladium (II) (12 mg, 0.016 mmol) and K₂CO₃ (66 mg, 0.48 mmol). The mixture was degassed with argon for 10 min, then dioxane (2 mL) and water (0.5 mL) were added. The reaction was heated at 90° C. for 5 h. The reaction was cooled to rt, partitioned between EtOAc and water. The organic layers were dried over Na₂SO₄, concentrated, and purified by silica-gel column chromatography eluting with a gradient (0-10%) CH₂Cl₂/MeOH to afford 7-((3S,4S)-3-fluoro-2,2,6,6-tetramethylpiperidin-4-yl)-3-(2-(methoxymethoxy)-4-(4-methyl-1H-1,2,3-triazol-1-yl)phenyl)-6,7-dihydro-5H-pyrrolo[2,3-c]pyridazine (58 mg, 73% yield). MS m/z 496.5 [M+H]⁺.

Step 2. To a sealed tube were added 7-((3S,4S)-3-fluoro-2,2,6,6-tetramethylpiperidin-4-yl)-3-(2-(methoxymethoxy)-4-(4-methyl-1H-1,2,3-triazol-1-yl)phenyl)-6,7-dihydro-5H-pyrrolo[2,3-c]pyridazine (58 mg, 0.12 mmol), active MnO₂ (204 mg, 2.34 mmol) and anhydrous toluene (1.0 mL). This mixture was stirred at 90° C. for 7 h then cooled to room temperature. The solid materials were removed by filtration, and the filtrate was concentrated to give crude 7-((3S,4S)-3-fluoro-2,2,6,6-tetramethylpiperidin-4-yl)-3-(2-(methoxymethoxy)-4-(4-methyl-1H-1,2,3-triazol-1-yl)phenyl)-7H-pyrrolo[2,3-c]pyridazine (57 mg, 99% yield) as brown solid which was used in the next step without further purification. MS m/z 494.5 [M+H]⁺.

Step 3: A solution of 7-((3S,4S)-3-fluoro-2,2,6,6-tetramethylpiperidin-4-yl)-3-(2-(methoxymethoxy)-4-(4-methyl-1H-1,2,3-triazol-1-yl)phenyl)-7H-pyrrolo[2,3-c]pyridazine (57 mg, 0.12 mmol) in TFA (1 mL) was heated to 70° C. for 5 min. The mixture was concentrated, and the residue was purified by reverse phase chromatography eluting with a gradient ACN/H₂O/formic acid (0-100% ACN) to give 2-(7-((3S,4S)-3-fluoro-2,2,6,6-tetramethylpiperidin-4-yl)-7H-pyrrolo[2,3-c]pyridazin-3-yl)-5-(4-methyl-1H-1,2,3-triazol-1-yl)phenol (25 mg, 48% yield) as a tan solid. MS m/z 450.5 [M+H]⁺; ¹H NMR (methanol-d₄) δ: 8.80 (s, 1H), 8.36 (s, 2H), 8.02 (d, J=8.63 Hz, 1H), 7.60 (d, J=2.00 Hz, 2H), 7.04 (d, J=3.50 Hz, 1H), 5.81-6.07 (m, 1H), 5.03-5.24 (m, 1H), 2.79-2.84 (m, 1H), 2.44 (s, 3H), 2.31-2.40 (m, 1H), 1.73-1.86 (m, 6H), 1.57-1.72 (m, 6H); 2Hs not observed (NH and OH).

Using the procedure described for Example 3, above, additional compounds described herein may be prepared by substituting the appropriate starting material, suitable reagents and reaction conditions, obtaining compounds such as those selected from:

| Cpd | Data |
|---|---|
| 10 | MS m/z 450.4 [M + H]⁺; ¹H NMR (methanol-d₄) δ: 8.58-8.63 (m, 1 H), |
| | 8.46 (s, 1 H), 8.14 (d, J = 8.54 Hz, 1 H), 7.94 (s, 1 H), 7.44-7.54 (m, 2 |
| | H), 6.59 (s, 1 H), 5.64-5.80 (m, 1 H), 3.74 (t, J = 13.73 Hz, 2 H), 3.23 |
| | (q, J = 7.32 Hz, 1 H), 2.74 (s, 3 H), 2.34 ( dd, J = 13.73, 3.05 Hz, 1 H), |
| | 1.80 (s, 3 H), 1.75 (s, 3 H), 1.69 (s, 3 H), 1.59 (s, 3 H) |
| 16 | MS m/z 402.5 [M + H]⁺; ¹H NMR (methanol-d₄) δ: 8.65 (s, 1H), 8.34 (s, |
| | 1H), 8.23 (s, 1H), 8.16 (s, 1H), 8.01 (d, J = 3.5 Hz, 1H), 7.44-7.52 (m, |
| | 1H), 6.79 (d, J = 3.6 Hz, 1H), 5.29 (s, 1H), 3.60-3.69 (m, 1H), 3.41-3.56 |
| | (m, 1H), 3.06 (t, J = 12.9 Hz, 1H), 2.58-2.73 (m, 1H), 2.50 (br d, J = |
| | 13.9 Hz, 1H), 2.44 (s, 3H), 1.82-1.93 (m, 1H), 1.09-1.22 (m, 2H), 0.90- |
| | 1.07 (m, 2H); 2Hs not observed (NH and OH) |
| 17 | MS m/z 450.5 [M + H]⁺; ¹H NMR (methanol-d₄) δ: 8.71 (s, 1H), 8.21 (d, |
| | J = 8.26 Hz, 1H), 8.09 (br d, J = 1.25 Hz, 1H), 7.69 (s, 1H), 7.20 (s, |
| | 2H), 6.85 (d, J = 3.50 Hz, 1H), 5.85-6.13 (m, 1H), 5.01-5.18 (m, 1H), |
| | 3.29-3.33 (m, 3H), 2.71-2.88 (m, 1H), 2.25-2.37 (m, 1H), 1.70-1.86 (m, |
| | 6H), 1.60-1.69 (m, 6H); 2Hs not observed (NH and OH) |
| 18 | MS m/z 402.5 [M + H]⁺; ¹H NMR (methanol-d₄) δ: 8.67 (s, 1H), 8.20 (d, |
| | J = 8.38 Hz, 1H), 8.02 (d, J = 3.50 Hz, 1H), 7.69 (s, 1H), 7.12-7.23 (m, |
| | 2H), 6.80 (d, J = 3.50 Hz, 1H), 5.30 (m, 1H), 3.64-3.76 (m, 1H), 3.42- |
| | 3.58 (m, 1H), 3.10 (m, 1H), 2.68 (m, 1H), 2.49-2.58 (m, 1H), 2.46 (s, |
| | 3H), 1.81-1.94 (m, 1H), 1.11-1.27 (m, 3H), 0.96-1.05 (m, 1H); 2Hs not |
| | observed (NH and OH) |
| 19 | MS m/z 470.2 [M + H]⁺ |

### Biological Examples

The following in vitro biological examples demonstrate the usefulness of the compounds of the present description for treating Huntington's disease.

To describe in more detail and assist in understanding the present description, the following non-limiting biological examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed.

Compounds of Formula (I) were tested using the Meso Scale Discovery (MSD) Assay provided in International Application No. PCT/US2016/066042, filed on Dec. 11, 2016 and claiming priority to United States Provisional Application U.S. 62/265,652 filed on Dec. 10, 2015, the entire contents of which are incorporated herein by reference.

The Endogenous Huntingtin Protein assay used in Example 1 was developed using the ELISA-based MSD electrochemiluminescence assay platform.

### Example 1

### Endogenous Huntingtin Protein Assay

Meso Scale Discovery (MSD) 96-well or 384-well plates were coated overnight at 4° C. with MW1 (expanded polyglutamine) or MAB2166 monoclonal antibody (for capture) at a concentration of 1 μg/mL in PBS (30 μL per well). Plates were then washed three times with 300 μL wash buffer (0.05% Tween-20 in PBS) and blocked (100 μL blocking buffer; 5% BSA in PBS) for 4-5 hours at room temperature with rotational shaking and then washed three times with wash buffer.

Samples (25 μL) were transferred to the antibody-coated MSD plate and incubated overnight at 4° C. After removal of the lysates, the plate was washed three times with wash buffer, and 25 μL of #5656S (Cell signaling; rabbit monoclonal) secondary antibody (diluted to 0.25 μg/mL in 0.05% Tween-20 in blocking buffer) was added to each well and incubated with shaking for 1 Hour at room temperature. Following incubation with the secondary antibody, the wells were rinsed with wash buffer after which 25 μL of goat anti-rabbit SULFO TAG secondary detection antibody (required aspect of the MSD system) (diluted to 0.25 μg/mL in 0.05% Tween-20 in blocking buffer) was added to each well and incubated with shaking for 1 hour at room temperature. After rinsing three times with wash buffer, 150 μL of read buffer T with surfactant (MSD) were added to each empty well, and the plate was imaged on a SI 6000 imager (MSD) according to manufacturers' instructions provided for 96- or 384-well plates. The resulting IC₅₀ values (μM) for compounds tested are shown in Table 1.

As shown in Table 1, test compounds described herein had the following IC₅₀ values, an IC₅₀ value between >3 μM and ≤9 μM is indicated by a single star (*), an IC₅₀ value between >1 μM and ≤3 μM is indicated by two stars (**), an IC₅₀ value between >0.5 M and ≤1 μM is indicated by three stars (***), an IC₅₀ value between >0.1 μM and ≤0.5 μM is indicated by four stars (****) and an IC₅₀ value of ≤0.1 μM is indicated by five stars (*****).

TABLE 1

| Cpd | IC₅₀ |
|---|---|
| 1 | ***** |
| 2 | ***** |
| 3 | ***** |
| 4 | ***** |
| 5 | ***** |
| 6 | *** |
| 7 | ***** |
| 8 | ***** |
| 9 | **** |
| 10 | ***** |
| 11 | ***** |
| 12 | ***** |
| 13 | ***** |
| 14 | ***** |
| 15 | ***** |
| 16 | ***** |
| 17 | **** |
| 18 | *** |
| 19 | ***** |
| 20 | ***** |
| 21 | ***** |
| 22 | ***** |
| 23 | ***** |
| 24 | ***** |
| 25 | **** |
| 26 | ***** |
| 27 | ***** |

### Example 2

### Results for Comparison Compounds: Improvement in Potency

Comparison Compounds were reported in International Publication No. WO2020/005873 as compounds which were found to have activity in the Endogenous Huntingtin Protein Assay. The Comparison Compounds lack various structural features compared to compounds of the invention encompassed by Formula (I). Comparison Compounds were tested according to the assay described in Example 1, and the results are shown in Table 2. It was observed that structural modifications resulted in divergent changes in potency.

A significant 223-fold improvement in potency was observed with Cpd 1 of the invention, having a 7H-pyrrolo[2,3-c]pyridazine core, compared with Cpd 72 in WO2020/005873, having a 3H-[1,2,3]-triazolo[4,5-c]pyridazine core. In contrast, similar potencies were observed with Cpd 26 in WO2020/005873, having a 7H-pyrrolo[2,3-c]pyridazine core, and Cpd 7 in WO2020/005873, having a 3H-[1,2,3]-triazolo[4,5-c]pyridazine core.

Comparison of Cpd 7 and Cpd 72 in WO2020/005873 shows that a significant loss in potency is observed when a 1H-pyrazole moiety is replaced with a 1H-1,2,3-triazole moiety. In contrast, an over 8-fold improvement in potency was observed between Cpd 1 of the invention and Cpd 26 in WO2020/005873 with the same structural modification.

TABLE 2

| Compound | IC₅₀ |
|---|---|
| 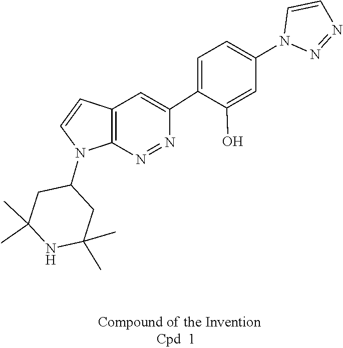 [SMILES: CC1(C)CC(n2ccc3cc(-c4ccc(-n5ccnn5)cc4O)nnc32)CC(C)(C)N1] | 0.8 nM |
| 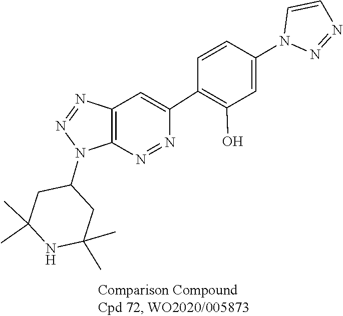 [SMILES: CC1(C)CC(n2nnc3cc(-c4ccc(-n5ccnn5)cc4O)nnc32)CC(C)(C)N1] | 179 nM |
| 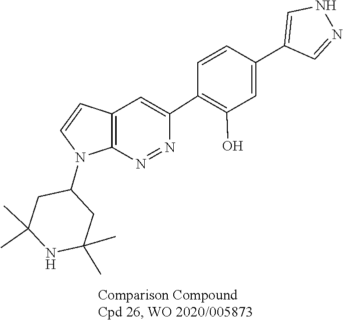 [SMILES: CC1(C)CC(n2ccc3cc(-c4ccc(-c5cn[nH]c5)cc4O)nnc32)CC(C)(C)N1] | 7.1 nM |
| 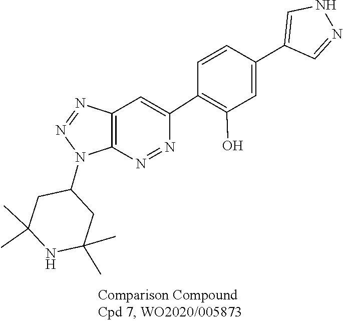 [SMILES: CC1(C)CC(n2nnc3cc(-c4ccc(-c5cn[nH]c5)cc4O)nnc32)CC(C)(C)N1] | 6.4 nM |

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or particular aspects described herein. It is intended that the appended claims be interpreted to include all such equivalents.

1. A compound of Formula (b): 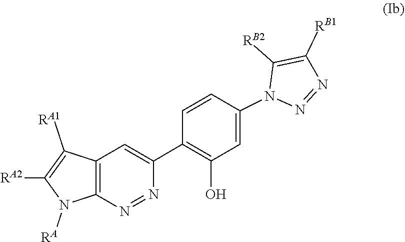
or a form thereof, wherein;
R^{A} is selected from the group consisting of: 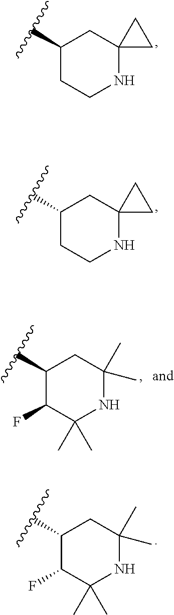 [SMILES: *[C@@H]1CCNC2(CC2)C1 ; *[C@H]1CCNC2(CC2)C1 ; *[C@H]1CC(C)(C)NC(C)(C)[C@H]1F ; *[C@@H]1CC(C)(C)NC(C)(C)[C@@H]1F]
R^{A1} and R^{A2} are each independently selected from the group consisting of hydrogen, deuterium, halogen, hydroxy, cyano, C₁₋₄alkyl, deutero-C₁₄alkyl, haloC₁₋₄alkyl, C₁₋₄alkoxy, haloC₁₋₄alkoxy, C₁₋₄alkoxyC₁₋₄alkyl, amino, C₁₋₄alkylamino, (C₁₋₄alkyl)₂amino, aminoC₁₋₄alkyl, and hydroxyC₁₋₄alkyl; and
R^{B1} and R^{B2} are each independently selected from the group consisting of hydrogen, deuterium, halogen, hydroxyl, cyano, C₁₋₄alkyl, deutero-C₁₋₄alkyl, halo-C₁₋₄alkyl, C₁₋₄alkoxy, deutero-C₁₋₄alkoxy, and halo-C₁₋₄alkoxy;
wherein the form of the compound is a salt form thereof.

2. (canceled)

3. (canceled)

4. (canceled)

5. (canceled)

6. (canceled)

7. (canceled)

8. (canceled)

9. The compound of claim 1, wherein the form of the compound is a salt selected from the group consisting of a hydrochloride salt, a dihydrochloride salt, a formate salt, a diformate salt, and a trifluroacetate salt.

10. The compound of claim 1, or a form thereof, selected from the group consisting of:
2-[7-(2,2,6,6-tetramethylpiperidin-4-yl)-7H-pyrrolo[2,3-c]pyridazin-3-yl]-5-(1H-1,2,3-triazol-1-yl)phenol;
2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol;
2-{7-[(3R,4R)-3-fluoro-2,2,6,6-tetramethylpiperidin-4-yl]-7H-pyrrolo[2,3-c]pyridazin-3-yl}-5-(1H-1,2,3-triazol-1-yl)phenol;
2-{7-[(3R,4R)-3-fluoro-2,2,6,6-tetramethylpiperidin-4-yl]-7H-pyrrolo[2,3-c]pyridazin-3-yl}-5-(1H-1,2,3-triazol-1-yl)phenol;
2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol;
2-{7-[(3R,4S)-3-fluoro-2,2,6,6-tetramethylpiperidin-4-yl]-7H-pyrrolo[2,3-c]pyridazin-3-yl}-5-(1H-1,2,3-triazol-1-yl)phenol;
2-{7-[(4RS)-2,2-dimethylpiperidin-4-yl]-7H-pyrrolo[2,3-c]pyridazin-3-yl}-5-(1H-1,2,3-triazol-1-yl)phenol;
5-(1H-1,2,3-triazol-1-yl)-2-{7-[(4RS)-1,2,2-trimethylpiperidin-4-yl]-7H-pyrrolo[2,3-c]pyridazin-3-yl}phenol;
2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol);
2-{7-[(3S,4S)-3-fluoro-2,2,6,6-tetramethylpiperidin-4-yl]-6-methyl-7H-pyrrolo[2,3-c]pyridazin-3-yl}-5-(1H-1,2,3-triazol-1-yl)phenol;
2-{7-[(4S,5R)-5-fluoro-2,2-dimethylpiperidin-4-yl]-7H-pyrrolo[2,3-c]pyridazin-3-yl}-5-(1H-1,2,3-triazol-1-yl)phenol;
2-{7-[(4R,5S)-5-fluoro-2,2-dimethylpiperidin-4-yl]-7H-pyrrolo[2,3-c]pyridazin-3-yl}-5-(1H-1,2,3-triazol-1-yl)phenol;
2-{7-[(3S,4S)-3-fluoro-2,2,6,6-tetramethylpiperidin-4-yl]-7H-pyrrolo[2,3-c]pyridazin-3-yl}-5-(4-methyl-1H-1,2,3-triazol-1-yl)phenol;
2-{7-[(3S,4S)-3-fluoro-2,2,6,6-tetramethylpiperidin-4-yl]-7H-pyrrolo[2,3-c]pyridazin-3-yl}-5-(5-methyl-1H-1,2,3-triazol-1-yl)phenol;
5-(4-chloro-1H-1,2,3-triazol-1-yl)-2-{7-[(3S,4S)-3-fluoro-2,2,6,6-tetramethylpiperidin-4-yl]-7H-pyrrolo[2,3-c]pyridazin-3-yl}phenol;
2-{7-[(1S,2S,3R,5R)-2-fluoro-8-azabicyclo[3.2.1]octan-3-yl]-7H-pyrrolo[2,3-c]pyridazin-3-yl}-5-(1H-1,2,3-triazol-1-yl)phenol;
2-{7-[(1R,2R,3S,5S)-2-fluoro-8-azabicyclo[3.2.1]octan-3-yl]-7H-pyrrolo[2,3-c]pyridazin-3-yl}-5-(1H-1,2,3-triazol-1-yl)phenol;
2-{7-[(1S,2R,3R,5R)-2-fluoro-1,5-dimethyl-8-azabicyclo[3.2.1]octan-3-yl]-7H-pyrrolo[2,3-c]pyridazin-3-yl}-5-(1H-1,2,3-triazol-1-yl)phenol;
2-{7-[(1R,2S,3S,5S)-2-fluoro-1,5-dimethyl-8-azabicyclo[3.2.1]octan-3-yl]-7H-pyrrolo[2,3-c]pyridazin-3-yl}-5-(1H-1,2,3-triazol-1-yl)phenol;
2-{7-[(4S,5R)-5-fluoro-2,2-dimethylpiperidin-4-yl]-7H-pyrrolo[2,3-c]pyridazin-3-yl}-5-(1H-1,2,3-triazol-1-yl)phenol;
2-[7-(4-azadispiro[2.1.2⁵.3³]decan-9-yl)-7H-pyrrolo[2,3-c]pyridazin-3-yl]-5-(1H-1,2,3-triazol-1-yl)phenol;
4-fluoro-2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)pheno; and,
3-fluoro-2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol;
wherein the form of the compound is a salt form thereof.

11. The compound of claim 10, wherein the compound is a salt form selected from the group consisting of:
2-[7-(2,2,6,6-tetramethylpiperidin-4-yl)-7H-pyrrolo[2,3-c]pyridazin-3-yl]-5-(1H-1,2,3-triazol-1-yl)phenol dihydrochloride;
2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol dihydrochloride;
2-{7-[(3R,4R)-3-fluoro-2,2,6,6-tetramethylpiperidin-4-yl]-7H-pyrrolo[2,3-c]pyridazin-3-yl}-5-(1H-1,2,3-triazol-1-yl)phenol dihydrochloride;
2-{7-[(3R,4S)-3-fluoro-2,2,6,6-tetramethylpiperidin-4-yl]-7H-pyrrolo[2,3-c]pyridazin-3-yl}-5-(1H-1,2,3-triazol-1-yl)phenol dihydrochloride;
2-{7-[(3S,4R)-3-fluoro-2,2,6,6-tetramethylpiperidin-4-yl]-7H-pyrrolo[2,3-c]pyridazin-3-yl}-5-(1H-1,2,3-triazol-1-yl)phenol dihydrochloride;
2-{7-[(4RS)-2,2-dimethylpiperidin-4-yl]-7H-pyrrolo[2,3-c]pyridazin-3-yl}-5-(1H-1,2,3-triazol-1-yl)phenol hydrochloride;
5-(1H-1,2,3-triazol-1-yl)-2-{7-[(4RS)-1,2,2-trimethylpiperidin-4-yl]-7H-pyrrolo[2,3-c]pyridazin-3-yl}phenol hydrochloride;
2-{7-[(3S,4S)-3-fluoro-2,2,6,6-tetramethylpiperidin-4-yl]-7H-pyrrolo[2,3-c]pyridazin-3-yl}-5-(1H-1,2,3-triazol-1-yl)phenol dihydrochloride;
2-{7-[(3S,4S)-3-fluoro-2,2,6,6-tetramethylpiperidin-4-yl]-6-methyl-7H-pyrrolo[2,3-c]pyridazin-3-yl}-5-(1H-1,2,3-triazol-1-yl)phenol diformate;
2-{7-[(4S,5R)-5-fluoro-2,2-dimethylpiperidin-4-yl]-7H-pyrrolo[2,3-c]pyridazin-3-yl}-5-(1H-1,2,3-triazol-1-yl)phenol hydrochloride;
2-{7-[(4R,5S)-5-fluoro-2,2-dimethylpiperidin-4-yl]-7H-pyrrolo[2,3-c]pyridazin-3-yl}-5-(1H-1,2,3-triazol-1-yl)phenol hydrochloride;
2-{7-[(3S,4S)-3-fluoro-2,2,6,6-tetramethylpiperidin-4-yl]-7H-pyrrolo[2,3-c]pyridazin-3-yl}-5-(4-methyl-1H-1,2,3-triazol-1-yl)phenol formate;
2-{7-[(3S,4S)-3-fluoro-2,2,6,6-tetramethylpiperidin-4-yl]-7H-pyrrolo[2,3-c]pyridazin-3-yl}-5-(5-methyl-1H-1,2,3-triazol-1-yl)phenol formate;
5-(4-chloro-1H-1,2,3-triazol-1-yl)-2-{7-[(3S,4S)-3-fluoro-2,2,6,6-tetramethylpiperidin-4-yl]-7H-pyrrolo[2,3-c]pyridazin-3-yl}phenol formate.

12. A method for treating or ameliorating HD in a subject in need thereof, comprising administering to a subject an effective amount of the compound of claim 1.

13. The method of claim 12, wherein the effective amount of the compound is in a range of from about 0.001 mg/kg/day to about 500 mg/kg/day.

14. A use of the compound of claim 1 for treating or ameliorating HD in a subject in need thereof, comprising administering to the subject an effective amount of the compound.

15. The use of claim 14, wherein the effective amount of the compound is in a range of from about 0.001 mg/kg/day to about 500 mg/kg/day.

16. A use of the compound of claim 1 in the manufacture of a medicament for treating or ameliorating HD in a subject in need thereof, comprising administering an effective amount of the medicament to the subject.

17. The use of claim 16, wherein the effective amount of the compound in the medicament is in a range of from about 0.001 mg/kg/day to about 500 mg/kg/day.

18. A use of the compound of claim 1 in admixture with one or more pharmaceutically acceptable excipient(s) in a pharmaceutical composition for treating or ameliorating HD in a subject in need thereof, comprising administering an effective amount of the pharmaceutical composition to the subject.

19. The use of claim 18, wherein the effective amount of the compound in the pharmaceutical composition is in a range of from about 0.001 mg/kg/day to about 500 mg/kg/day.

20. A pharmaceutical composition for use in treating or ameliorating HD comprising an effective amount of the compound of claim 1 in admixture with one or more pharmaceutically acceptable excipient(s).

21. A pharmaceutical composition for use in treating or ameliorating HD comprising an effective amount of the compound of claim 10 in admixture with one or more pharmaceutically acceptable excipient(s).

22. The compound of claim 1, or a form thereof, selected from the group consisting of:
2-[7-(4-azaspiro[2.5]octan-7-yl)-7H-pyrrolo[2,3-c]pyridazin-3-yl]-5-(1H-1,2,3-triazol-1-yl)phenol;
2-[7-(4-methyl-4-azaspiro[2.5]octan-7-yl)-7H-pyrrolo[2,3-c]pyridazin-3-yl]-5-(1H-1,2,3-triazol-1-yl)phenol;
2-[7-(4-azaspiro[2.5]octan-7-yl)-6-methyl-7H-pyrrolo[2,3-c]pyridazin-3-yl]-5-(1H-1,2,3-triazol-1-yl)phenol;
2-{7-[(7R)-4-azaspiro[2.5]octan-7-yl]-7H-pyrrolo[2,3-c]pyridazin-3-yl}-5-(1H-1,2,3-triazol-1-yl)phenol;
2-{7-[(7S)-4-azaspiro[2.5]octan-7-yl]-7H-pyrrolo[2,3-c]pyridazin-3-yl}-5-(1H-1,2,3-triazol-1-yl)phenol;
2-{7-[(7S)-4-azaspiro[2.5]octan-7-yl]-7H-pyrrolo[2,3-c]pyridazin-3-yl}-5-(1H-1,2,3-triazol-1-yl)pyridine-3-ol;
2-{7-[(7S)-4-azaspiro[2.5]octan-7-yl]-7H-pyrrolo[2,3-c]pyridazin-3-yl}-5-(4-methyl-1H-1,2,3-triazol-1-yl)phenol;
2-{7-[(7S)-4-azaspiro[2.5]octan-7-yl]-7H-pyrrolo[2,3-c]pyridazin-3-yl}-5-(5-methyl-1H-1,2,3-triazol-1-yl)phenol;
2-{7-[(8R,9R)-9-fluoro-5-azaspiro[3.5]nonan-8-yl]-7H-pyrrolo[2,3-c]pyridazin-3-yl}-5-(1H-1,2,3-triazol-1-yl)phenol;
2-{7-[(8S,9S)-9-fluoro-5-azaspiro[3.5]nonan-8-yl]-7H-pyrrolo[2,3-c]pyridazin-3-yl}-5-(1H-1,2,3-triazol-1-yl)phenol;
2-{7-[(8S,9R)-8-fluoro-6-azaspiro[4.5]decan-9-yl]-7H-pyrrolo[2,3-c]pyridazin-3-yl}-5-(1H-1,2,3-triazol-1-yl)phenol; and,
2-{7-[(8R,9S)-8-fluoro-6-azaspiro[4.5]decan-9-yl]-7H-pyrrolo[2,3-c]pyridazin-3-yl}-5-(1H-1,2,3-triazol-1-yl)phenol;
wherein the form of the compound is a salt form thereof.

23. The compound of claim 22, wherein the compound is a salt form selected from the group consisting of:
2-[7-(4-azaspiro[2.5]octan-7-yl)-7H-pyrrolo[2,3-c]pyridazin-3-yl]-5-(1H-1,2,3-triazol-1-yl)phenol dihydrochloride;
2-[7-(4-methyl-4-azaspiro[2.5]octan-7-yl)-7H-pyrrolo[2,3-c]pyridazin-3-yl]-5-(1H-1,2,3-triazol-1-yl)phenol hydrochloride;
2-[7-(4-azaspiro[2.5]octan-7-yl)-6-methyl-7H-pyrrolo[2,3-c]pyridazin-3-yl]-5-(1H-1,2,3-triazol-1-yl)phenol diformate;
2-{7-[(7S)-4-azaspiro[2.5]octan-7-yl]-7H-pyrrolo[2,3-c]pyridazin-3-yl}-5-(1H-1,2,3-triazol-1-yl)phenol trifluoroacetate;
2-{7-[(7S)-4-azaspiro[2.5]octan-7-yl]-7H-pyrrolo[2,3-c]pyridazin-3-yl}-5-(1H-1,2,3-triazol-1-yl)pyridine-3-ol trifluoroacetate;
2-{7-[(7S)-4-azaspiro[2.5]octan-7-yl]-7H-pyrrolo[2,3-c]pyridazin-3-yl}-5-(4-methyl-1H-1,2,3-triazol-1-yl)phenol formate; and,
2-{7-[(7S)-4-azaspiro[2.5]octan-7-yl]-7H-pyrrolo[2,3-c]pyridazin-3-yl}-5-(5-methyl-1H-1,2,3-triazol-1-yl)phenol formate formate.

24. A pharmaceutical composition comprising an effective amount of the compound of claim 1 in admixture with one or more pharmaceutically acceptable excipient(s).

25. The pharmaceutical composition of any one of claim 24, wherein the effective amount of the compound is in a range of from about 0.001 mg/kg/day to about 500 mg/kg/day.
